(19) United States
(12) Patent Application Publication
(10) Pub. No.: US 2026/0355358 A1
(43) Pub. Date: Nov. 12, 2026

(54) HIGH RELAXIVITY CONTRAST AGENTS AND STEREOSELECTIVE PREPARATION

(71) Applicant: Portland State University, Portland, OR (US)

(72) Inventors: Mark Woods, Portland, OR (US); Karley B. Maier, Portland, OR (US); Lauren N. Rust, Portland, OR (US)

(73) Assignee: Portland State University, Portland, OR (US)

(21) Appl. No.: 19/099,003

(22) PCT Filed: Jul. 24, 2023
(86) PCT No.: PCT/US2023/070859

§ 371 Date: Jan. 27, 2025

Related U.S. Application Data
(60) Provisional application No. 63/394,133, filed on Aug. 1, 2022.

(51) Int. Cl. A61K 49/10; C07D 257/02; C07F 5/00

(52) U.S. Cl. CPC A61K 49/108; C07D 257/02; C07F 5/003

(57) ABSTRACT

A compound has a structure according to formula IA or IB, or a salt or stereoisomer thereof:

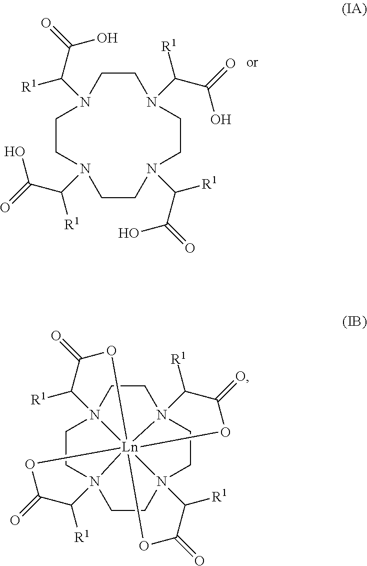

where each R¹ independently is an aryl or aliphatic group having an sp²-hybridized or sp-hybridized carbon at its attachment point to the carbon atom alpha to nitrogen, and Ln is a lanthanide ion. The aryl or aliphatic group may be substituted or unsubstituted. The disclosed compounds may be useful as contrast agents, such as magnetic resonance imaging contrast agents. Some stereoisomers of the disclosed chelated compounds according to formula IB have a high relaxivity. In certain instances, the relaxivity is close to a theoretical maximum value for the chelate. A stereoselective synthesis for preparing the compounds is disclosed.

### CROSS-REFERENCE TO RELATED APPLICATION

This application claims the benefit of the earlier filing date of U.S. Provisional Application No. 63/394,133, filed Aug. 1, 2023, which is incorporated by reference in its entirety herein.

### ACKNOWLEDGMENT OF GOVERNMENT SUPPORT

This invention was made with government support under R21 GM127964 awarded by the National Institutes of Health, and 1638278 awarded by the National Science Foundation. The government has certain rights in the invention.

### FIELD

This disclosure concerns contrast agents for magnetic resonance imaging, as well as methods of stereoselectively preparing the contrast agents.

### SUMMARY

Contrast agents for magnetic resonance imaging are disclosed, along with methods of making and using the contrast agents. Aspects of the disclosed contrast agents are compounds according to formula IB or IA, or salts or stereoisomers thereof:

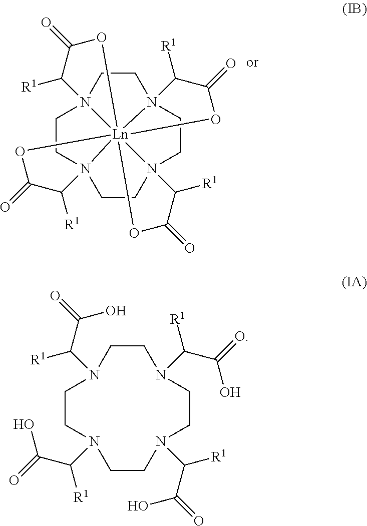

Each R¹ independently is an aryl or aliphatic group having an sp²-hybridized or sp-hybridized carbon at its attachment point to the carbon atom alpha to nitrogen; and Ln is a lanthanide ion.

In some aspects, each R¹ independently is aryl, alkenyl, or alkynyl. In any of the foregoing or following aspects, each R¹ independently may be aryl substituted with one or more substituents R², where each R² independently is —(CH₂)ₙC(O)OR^{a}, —(CH₂)ₙOR^{a}, —(CH₂)ₙOC(O)R^{a}, —(CH₂)ₙC(O)R^{a}, —(CH₂)ₙC(S)R^{a}, —(CH₂)ₙCOR^{a}, —(CH₂)ₙCSR^{a}, —(CH₂)ₙSR^{a}, —(CH₂)ₙNO₂, —(CH₂)ₙN(R^{b})R^{c}, —(CH₂)ₙC(O)N(R^{b})R^{c}, —(CH₂)ₙC(S)N(R^{b})R^{c}, —(CH₂)ₙCN, alkyl, alkenyl, alkynyl, or halo, where each R^{a}, R^{b}, and R^{c} independently is H or alkyl, and n is an integer from 0 to 10. In certain examples, each R¹ independently is

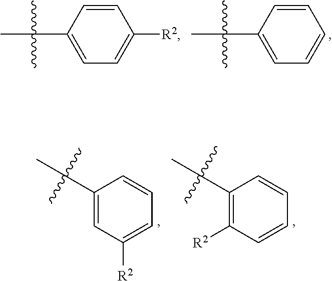

unsubstituted naphthalenyl, or alkenyl. Exemplary R¹ groups include

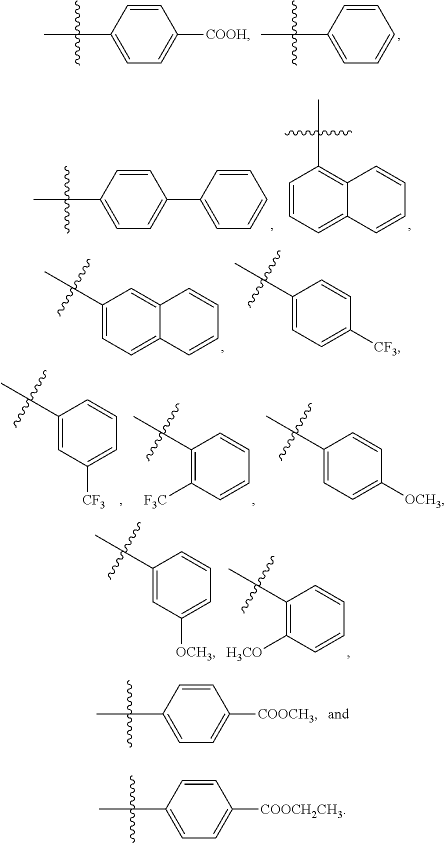

In certain aspects, each R¹ is the same.

In any of the foregoing or following aspects, the compound according to formula IB may be diastereoisomer, and molecules of the diastereomer may comprise a mixture of RRRR- and SSSS-enantiomers. In some aspects, at least 70% of molecules of the compound independently are RRRR- or SSSS-enantiomers; and a solution of the compound in water has a relaxivity r₁≥6 mM⁻¹s⁻¹ at a nuclear magnetic resonance field strength of 0.5 T to 7 T and 310K. In certain examples, the compound is:

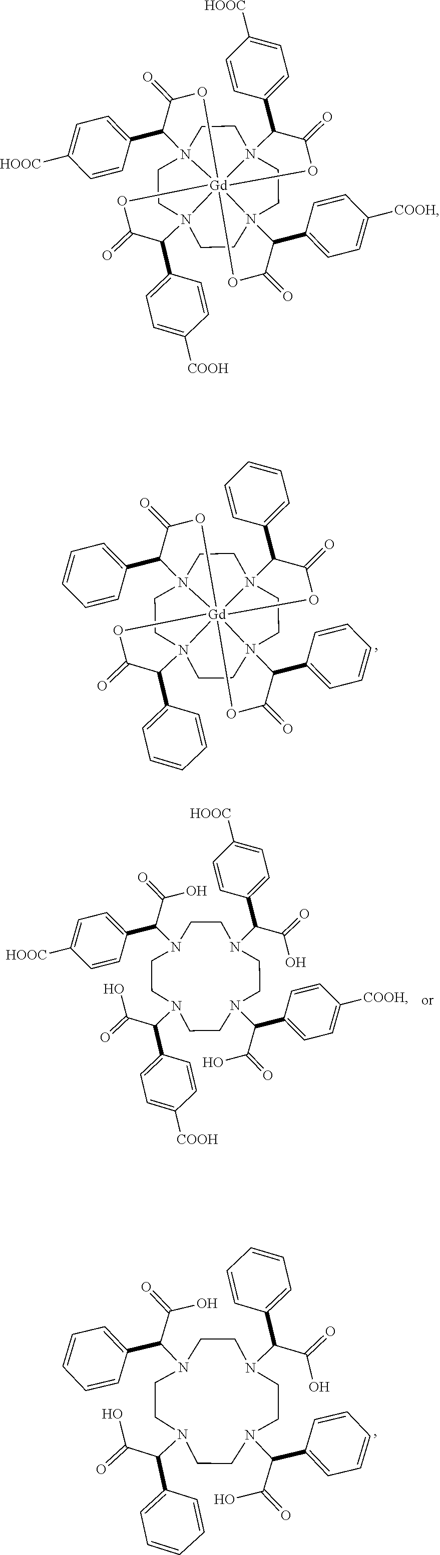

Aspects of a pharmaceutical composition include a compound according to formula IB and a pharmaceutically acceptable carrier. In some implementations, the compound is a diastereomer and at least 70% of molecules of the compound independently are RRRR- or SSSS-enantiomers. In certain examples, the compound is:

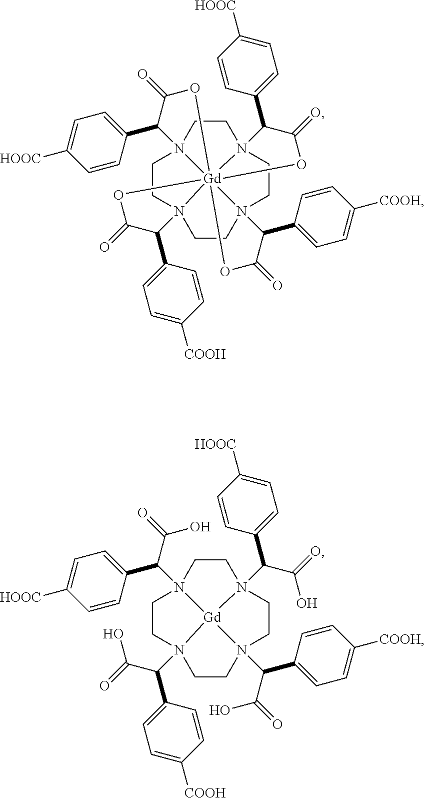

or a combination thereof.

Aspects of making and using the compounds also are disclosed.

### BRIEF DESCRIPTION OF THE DRAWINGS

FIG. 1 is a ¹H NMR spectrum, focused on the most highly shifted ax^{S} resonances, of ±EuDOTMA recorded at 600 MHz in D₂O at pD 6.

FIG. 2 shows the ¹H resonance of the chiral proton of R-methyl α-bromo phenylacetate R-2 prepared from R-phenylglycine in the absence (bottom, blue) and presence (top, red) of Eu(−)hfc₃ at 400 MHz in CDCl₃.

FIG. 3 is a diagram showing all possible sequences of substitution in a racemic synthesis of a tetra-α-substituted DOTA derivative after the first alkylation step has afforded an R-configuration.

FIGS. 4A-4D show an analysis of chelates produced in the synthesis of EuDOTFA. FIG. 4A is the chromatogram from the RP-HPLC separation of crude EuDOTFA (C18 column, λ—254 nm). FIG. 4B is the ¹H NMR spectrum (focused on the most shifted ax^{S} protons) of the first peak to elute, which appears to be the RSRS-isomer. FIG. 4C is the ¹H NMR spectrum (focused on the most shifted ax^{S} protons) of the second peak to elute, which appears to be the RRRS-/SSSR-isomer. FIG. 4D is the ¹H NMR spectrum (focused on the most shifted ax^{S} protons) of the third peak to elute, which is the RRRR-/SSSS-isomer. NMR spectra were recorded at 400 MHz in D₂O.

FIG. 5 shows the carbonyl region of the 13C NMR spectrum (600 MHz, D₂O) of H₈DOTBA.

FIG. 6 shows ¹H NMR spectra (focused on the most shifted ax^{S} region of the spectrum) of samples of EuDOTFA (left) and EuDOTBA (right) both prepared from ligand samples that contained a mixture of diastereoisomers.

FIG. 7 shows partial ¹H NMR spectra (600 MHz) of EuDOTBA after incubation in D₂O at pD 11, 60 °C focusing on the most shifted resonances of the SAP isomer: left, axial proton of the side carbon (ax^{S}) which serves as a control; right, the chiral methyne proton which was the anticipated point of alkylation.

FIG. 8 shows the up-field (right) and down-field (left) regions of the ¹H NMR spectra recorded during the chelation of Eu³⁺ with ±DOTBA in D₂O at pD 13, recorded at 600 MHz. A reference spectrum of the RRRR-/SSSS-isomer is shown (top).

FIG. 9 is a graph showing proportions of each stereoisomer produced as a function of the pH at which the chelation reaction was performed to provide EuDOTFA.

FIGS. 10A-10B show the temperature dependence of the 170 reduced transverse relaxation rate constant of solvent water of solutions of GdDOTFA (FIG. 10A) and GdDOTBA (FIG. 10B) at pH 6.6 and 11.7 T.

FIG. 11 shows ¹H nuclear magnetic relaxation dispersion (NMRD) profiles, recorded at 298K of GdDOTFA (open circles) and GdDOTBA (closed diamonds). For reference, the fit of the ¹H NMRD profile of GdDOTA is shown (dashed line).

FIGS. 12A and 12B are graphs showing relaxometric titrations at 298 K and 0.47 T of human serum albumin into 0.09 mM and 0.10 mM solutions of GdDOTFA (FIG. 12A) and GdDOBTA (FIG. 12B).

FIGS. 13A and 13B show the calculated relaxivity, as function of the water exchange lifetime (τ_{M}), of a Gd³⁺ chelate with electronic relaxation characteristics comparable to those of GdDOTA (Δ²=1.6×10⁻¹⁹ s⁻², τ_{V}=7.7 ps) if the rate of molecular tumbling had been slowed such that τ_{R}=100 ns (FIG. 13A) and τ_{R}=2 ns (FIG. 13B) at different magnetic field strengths. When TR is long, the value of τ_{M} is shorter at higher magnetic fields.

FIGS. 14A and 14B show the calculated relaxivity, as function of the water exchange lifetime (τ_{M}), of a Gd³⁺ chelate with electronic relaxation characteristics comparable to those of GdDOTFA (Δ²=6.8×10⁻¹⁸ s⁻², τ_{V}=25 ps) if the rate of molecular tumbled had been slowed such that τ_{R}=100 ns (FIG. 14A) and τ_{R}=2 ns (FIG. 14B) at different magnetic field strengths.

FIGS. 15A and 15B show calculated relaxivity as a function of time for GdDOTA (FIG. 15A) and GdDOTFA (FIG. 15B) incubated in 1 M HCl.

FIG. 16 shows chemical structures of Gd³⁺ chelates used as contrast agents that are either in clinical trials or already in clinical use.

FIG. 17 shows the relaxivity (expressed in terms of the total relaxivity per molecule) of GdDOTBA at 1.5 T (top) and 3.0 T (bottom) at 310 K. The relaxivities of clinically available contrast agents and another currently in trials under the same conditions are shown for comparative purposes.

FIG. 18 shows relaxivity (expressed in terms of the relaxivity per water molecule bound to Gd³⁺) of GdDOTBA at 1.5 T (top) and 3.0 T (bottom) at 310 K. The relaxivities of clinically available contrast agents and another currently in trials under the same conditions are shown for comparative purposes.

FIG. 19 shows NMR spectra of crude samples of four aryl-substituted EuDOTA chelates, focused on the most shifted axial proton resonance.

### DETAILED DESCRIPTION

This disclosure concerns compounds having a structure according to formula IA or IB, or a salt or stereoisomer thereof:

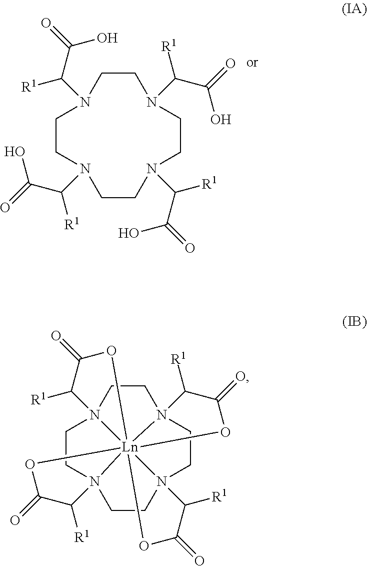

where each R¹ independently is an aryl or aliphatic group having an sp²-hybridized or sp-hybridized carbon at its attachment point to the carbon atom alpha to nitrogen, and Ln is a lanthanide ion. The disclosed compounds may be useful as contrast agents, such as magnetic resonance imaging contrast agents. Some stereoisomers of the disclosed chelated compounds according to formula IB have a high relaxivity. In certain instances, the relaxivity is close to a theoretical maximum value for the chelate. Embodiments of a stereoselective synthesis for preparing the compounds also are disclosed.

### I. DEFINITIONS AND ABBREVIATIONS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Additional definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). In order to facilitate review of the various embodiments of the disclosure, the following explanations of specific terms are provided:

Adduct: A product resulting from an addition reaction between two or more molecules. For example, molecules A and B react to form the product AB.

Aliphatic: A substantially hydrocarbon-based compound, or a radical thereof (e.g., C₆H₁₃, for a hexane radical), including alkanes, alkenes, alkynes, including cyclic versions thereof (also referred to as cycloaliphatic), and further including straight- and branched-chain arrangements, and all stereo and position isomers as well. Unless expressly stated otherwise, an aliphatic group contains from one to twenty-five carbon atoms; for example, from one to fifteen, from one to ten, from one to six, or from one to four carbon atoms. The term “lower aliphatic” refers to an aliphatic group containing from one to ten carbon atoms. An alkyl group is a hydrocarbon group having a saturated carbon chain. The chain may be cyclic, branched or unbranched. When the chain is cyclic, the group may be referred to as cycloalkyl. Examples, without limitation, of alkyl groups include methyl, ethyl, propyl, butyl, pentyl, hexyl, heptyl, octyl, nonyl and decyl. The term lower alkyl means the chain includes 1-10 carbon atoms. The terms alkenyl and alkynyl refer to hydrocarbon groups having carbon chains containing one or more double or triple bonds, respectively; cyclic versions may be referred to a cycloalkenyl or cycloalkynyl, respectively. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality. A substituted aliphatic group includes at least one sp³-hybridized carbon or two sp²-hybridized carbons bonded with a double bond or at least two sp-hybridized carbons bonded with a triple bond.

Aromatic: Unsaturated, cyclic hydrocarbons having alternate single and double bonds. Benzene, a 6-carbon ring containing three double bonds, is a typical aromatic compound.

Aryl: A monovalent aromatic carbocyclic group of, unless specified otherwise, from 6 to 15 carbon atoms having a single ring (e.g., phenyl) or multiple condensed rings in which at least one ring is aromatic (e.g., quinoline, indole, benzodioxole, and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Unless expressly referred to as an “unsubstituted aryl,” an aryl group can either be unsubstituted or substituted.

Chelate: A coordination compound in which a central metal ion is attached by coordinate bonds to two or more nonmetal atoms of a single molecule or ligand. The chelate may have a cyclic structure. As a verb, to form a chelate.

Coordination compound: A compound formed by a metal ion bonded to a non-metallic ion or molecule called a ligand. A coordinate bond is a covalent bond consisting of a pair of shared electrons donated by the ligand.

Effective amount with respect to a compound or composition refers to an amount of the compound or composition sufficient to achieve a particular desired result, e.g., detection during an imaging process.

Isomer: One of two or more molecules having the same number and kind of atoms, but differing in the arrangement or configuration of the atoms. Isomers that differ in the arrangement of their atoms in space are termed “stereoisomers”. Stereoisomers that are not mirror images of one another are termed “diastereomers” and those that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture.” As used herein, the term “coordination isomers” refers to isomers that differ in the geometry around a central coordinated atom. For example, embodiments of the disclosed chelates may be coordination isomers having a square antiprism (SAP) or twisted square antiprism (TSAP) geometry around a coordinated lanthanide ion.

Macromolecule: As used herein, the term “macromolecule” refers to a large molecule having a largest dimension (e.g., diameter) greater than 0.5 nm.

Pharmaceutically acceptable: A substance that can be taken into a subject without significant adverse toxicological effects on the subject.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more compositions. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some examples, the pharmaceutically acceptable carrier is a non-naturally occurring or synthetic carrier. The carrier also can be formulated in a unit-dosage form that carries a preselected therapeutic dosage of the active agent, for example in a pill, vial, bottle, or syringe.

Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.

Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Again solely by way of example and without limitation, a long-chain hydrocarbon may have a hydroxyl group bonded thereto.

### II. CONTRAST AGENTS

This disclosure concerns embodiments of contrast agents. In some embodiments, the contrast agents are useful for magnetic resonance imaging. Advantageously, the contrast agents may exhibit high relaxivity, e.g., ≥6 mM⁻¹s⁻¹ at a nuclear magnetic resonance field strength of 0.5 T to 7 T.

The disclosed compounds have a structure according to formula IA or IB, or a salt or stereoisomer thereof:

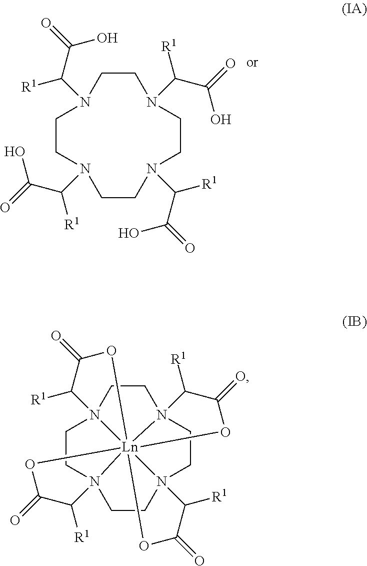

where each R¹ independently is an aryl or aliphatic group having an sp²-hybridized or sp-hybridized carbon at its attachment point to the carbon atom alpha to nitrogen; and Ln is a lanthanide ion. In some embodiments, the compound is optically active.

In any of the foregoing or following embodiments, each R¹ independently may be aryl, alkenyl, or alkynyl. Each R¹ may be substituted or unsubstituted. R¹ has an sp²-hybridized or sp-hybridized carbon at its attachment point to the carbon atom alpha to nitrogen. For example, if R¹ is butenyl, it may have a formula

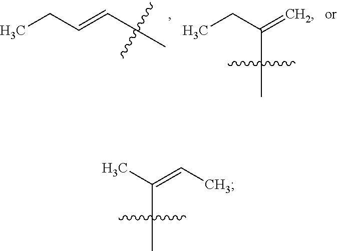

or if R¹ is propynyl, it has a formula

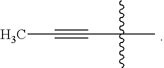 [SMILES: *C#CC]

In some embodiments, R¹ is substituted or unsubstituted aryl, such as substituted or unsubstituted phenyl, or substituted or unsubstituted naphthalenyl. In some implementations, R¹ is aryl substituted with one or more substituents R², where each R² independently is —(CH₂)ₙC(O)OR^{a}, —(CH₂)ₙOR^{a}, —(CH₂)ₙOC(O)R^{a}, —(CH₂)ₙC(O)R^{a}, —(CH₂)ₙC(S)R^{a}, —(CH₂)ₙCOR^{a}, —(CH₂)ₙCSR^{a}, —(CH₂)ₙSR^{a}, —(CH₂)ₙNO₂, —(CH₂)ₙN(R^{b})R^{c}, —(CH₂)ₙC(O)N(R^{b})R^{c}, —(CH₂)ₙC(S)N(R^{b})R^{c}, —(CH₂)ₙCN, alkyl, alkenyl, alkynyl, or halo (F, Cl, Br, or I), where each R^{a}, R^{b}, and R^{c} independently is H or alkyl, and n is an integer from 0 to 10. In some examples, n is 0, and R² is —C(O)OR^{a}, —OR^{a}, —OC(O)R^{a}, —C(O)R^{a}, —C(S)R^{a}, —COR^{a}, —CSR^{a}, —SR^{a}, —NO₂, —N(R^{b})R^{c}, —C(O)N(R^{b})R^{c}, —C(S)N(R^{b})R^{c}, —CN, alkyl, alkenyl, alkynyl, or halo. In certain examples, each R² independently is —C(O)OR^{a}, OR^{a}, alkyl, —SR^{a}, —NO₂, or halo. In some implementations, each R^{a}, R^{b}, and R^{c} independently is H or C₁-C₃ alkyl. In any of the foregoing or following aspects, an alkyl, alkenyl, or alkynyl group may be substituted or unsubstituted unless otherwise specified. In certain aspects, R¹ is unsubstituted phenyl, phenyl substituted with one or more R² groups, or unsubstituted naphthalenyl. Exemplary R² groups include, but are not limited to, —C(O)OR^{a}—OR^{a}, and alkyl, where R^{a} is H or C₁-C₃ alkyl. In some aspects, R² is —COOH, —OCH₃, —CF3, —COOCH₃, —COOCH₂CH₃(—COOEt), or phenyl.

In certain embodiments, R¹ is unsubstituted aryl or aryl substituted with one R² group. In some examples, the aryl group is a phenyl group or a naphthalenyl group. In some implementations, R¹ is unsubstituted phenyl,

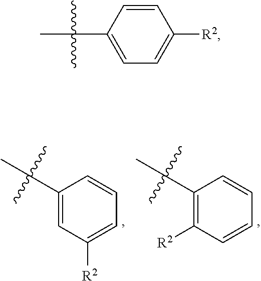

or unsubstituted naphthalenyl. Exemplary R¹ groups include, but are not limited to,

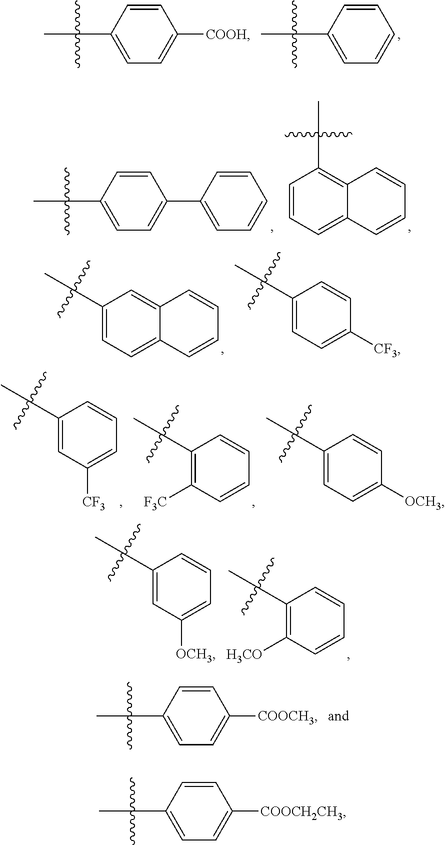

—CH═CHCOOH, and combinations thereof. In certain implementations, R¹ is

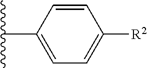

or unsubstituted phenyl. In particular examples, R¹ is

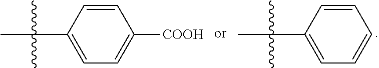 [SMILES: *c1ccc(C(=O)O)cc1 ; "or" ; *c1ccccc1]

In some embodiments, each R¹ is the same.

Ln is a lanthanide ion, such as Ln³⁺. The lanthanides include lanthanum and the elements with atomic numbers 58-71. In some embodiments, Ln is Gd or Eu. In particular implementations, Ln is Gd, and the compound according to formula IB is a Gd³⁺ chelate.

Exemplary compounds according to formula IA and IB include, but are not limited to:

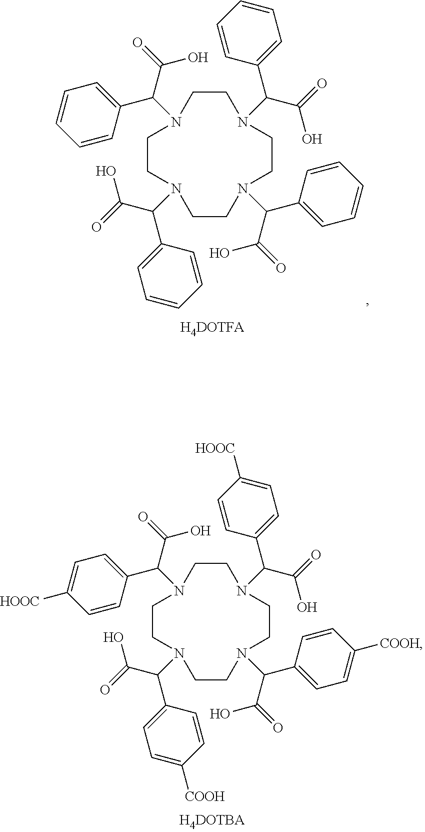

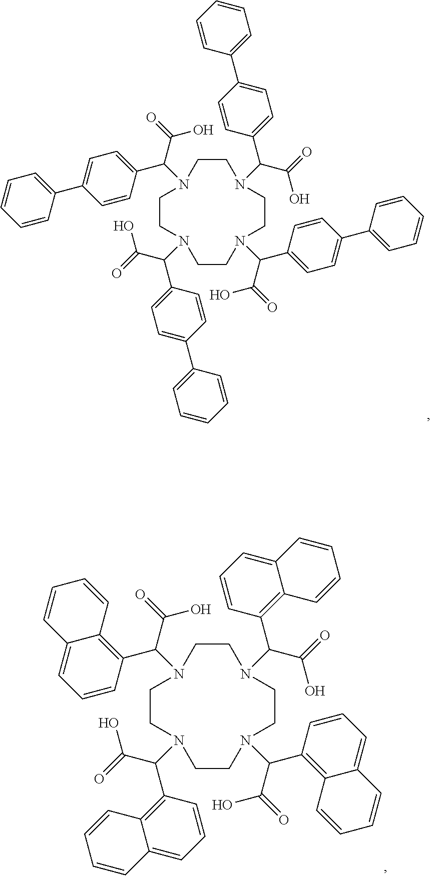

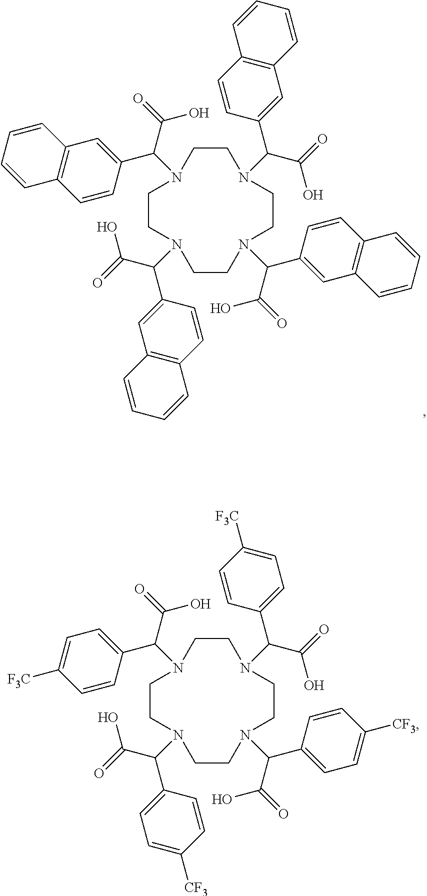

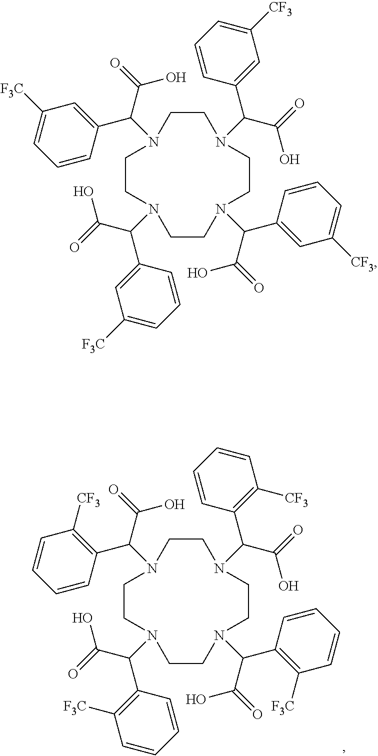

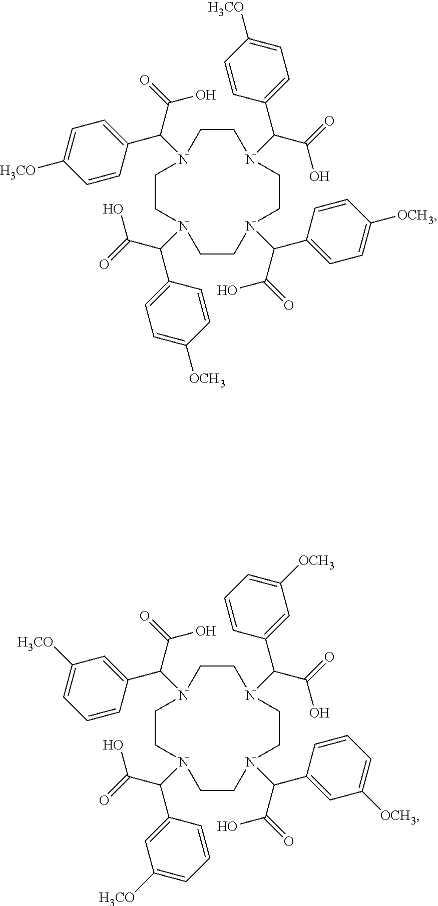

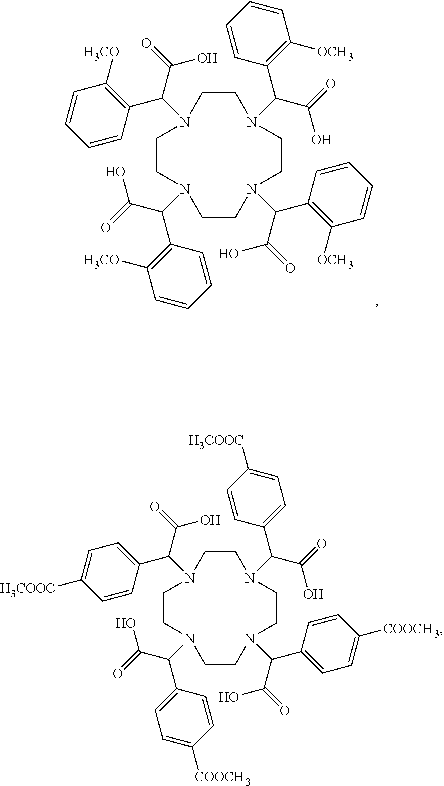

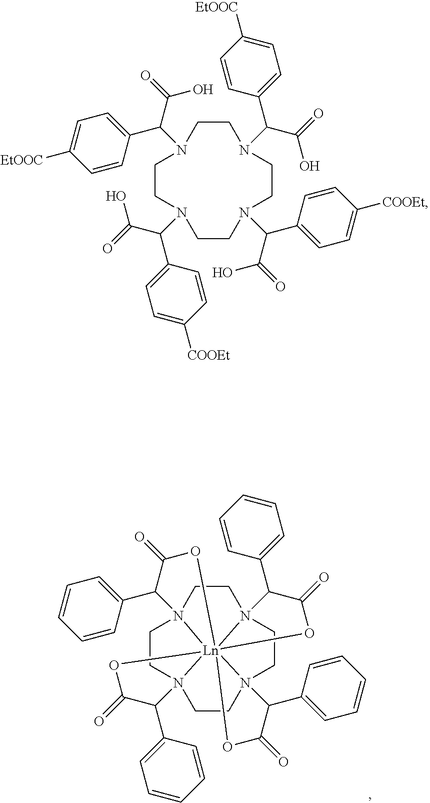

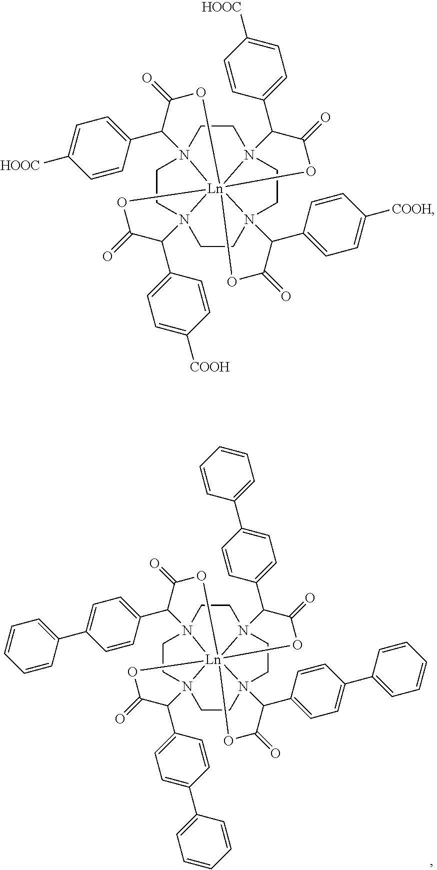

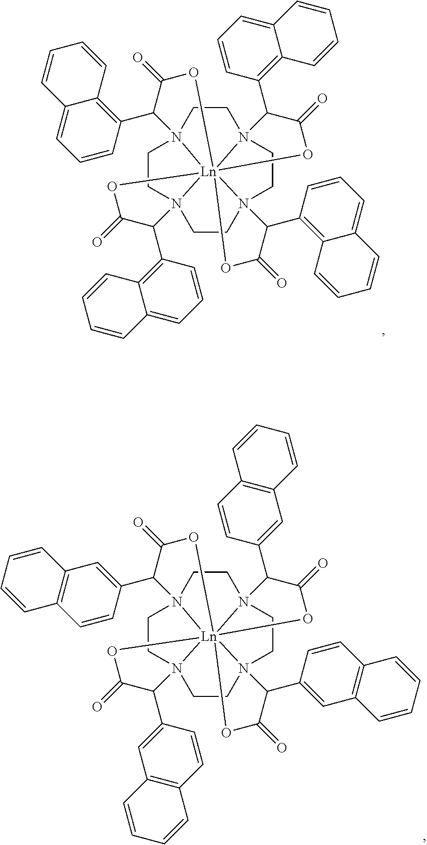

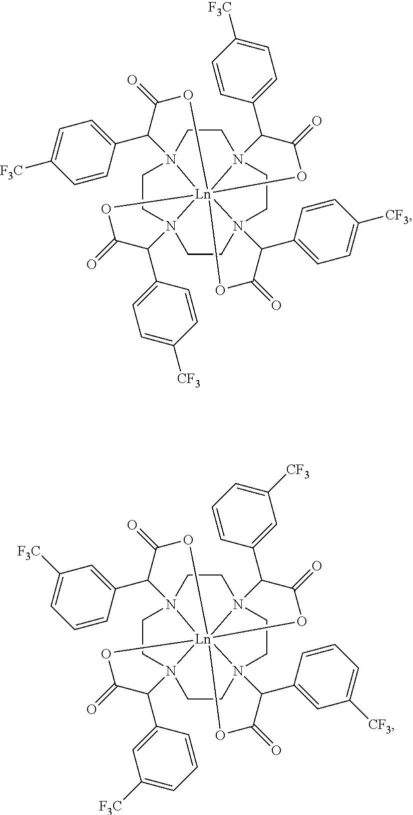

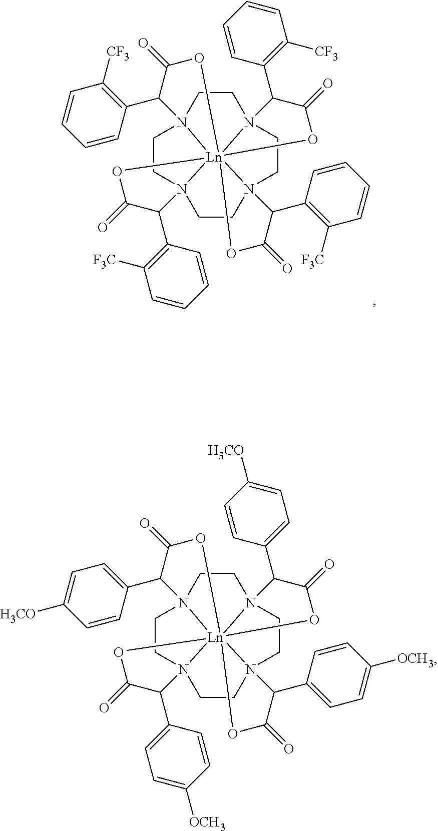

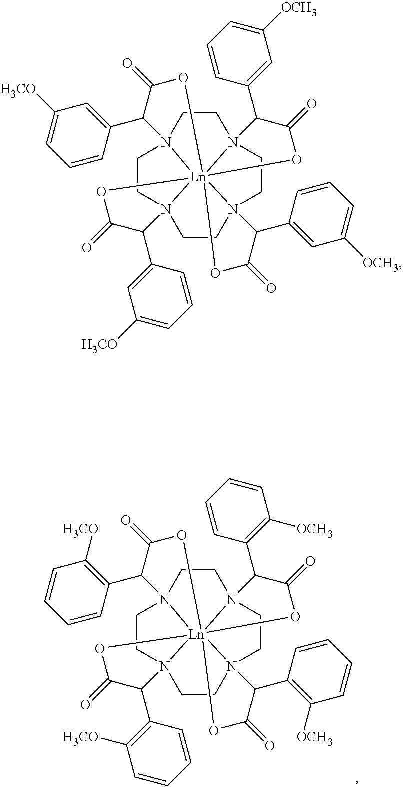

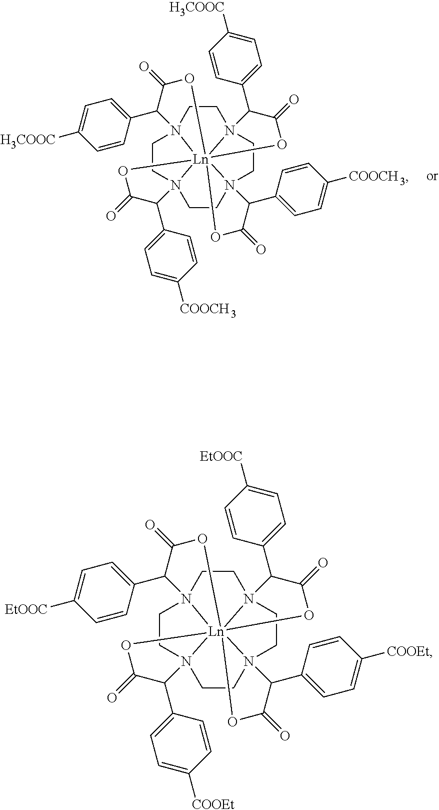

where Ln is Gd or Eu.

Embodiments of the disclosed compounds are stereoisomers with stereochemistry at the carbon atoms bonded to R¹ (i.e., stereochemistry at the carbon atoms alpha to the nitrogen atoms). With four stereocenters, RRRR-, SSSS-, RRRS-, SSSR-, RSRS-, and RRSS-isomers are possible. In some embodiments, the compound is a diastereomer, and molecules of the diastereomer comprise a mixture of RRRR- and SSSS-enantiomers. For example, the compound may be:

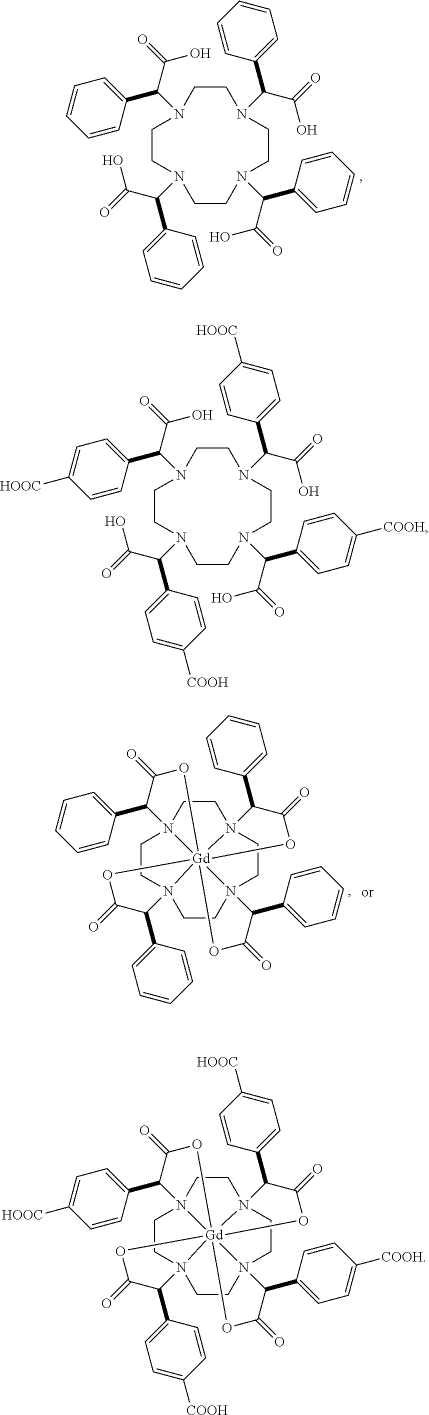

In any of the foregoing or following embodiments wherein at least 70% of molecules of the compound according to formula IB independently are RRRR- or SSSS-enantiomers, a solution of the compound in water may have a relaxivity r₁≥6 mM⁻¹s⁻¹ at a nuclear magnetic resonance field strength of 0.5 T to 7 T and 310K. In some embodiments, a solution of the compound in water has a relaxivity r₁ of ≥6 mM⁻¹s⁻¹, ≥7 mM⁻¹s⁻¹, or ≥8 mM⁻¹s⁻¹ at 0.5 T to 7 T and 310K, such as a relaxivity of 6 mM⁻¹s⁻¹ to 15 mM⁻¹s⁻¹, 7 mM⁻¹s⁻¹ to 12 mM⁻¹s⁻¹, or 8 mM⁻¹s⁻¹ to 10 mM⁻¹s⁻¹ at 0.5 T to 7 T and 310K.

This disclosure also includes pharmaceutical compositions comprising the disclosed contrast agents. In some embodiments, a pharmaceutical composition comprises a compound according to formula IB and a pharmaceutically acceptable carrier. The compound in the pharmaceutical composition may be a diastereoisomer with at least 70% of the compound molecules independently being RRRR- or SSSS-enantiomers. In some implementations, at least 75%, at least 80%, at least 85%, at least 90%, or at least 95% of the molecules independently are RRRR- or SSSS-enantiomers. The pharmaceutically acceptable carrier may be any conventional carrier. In some embodiments, the pharmaceutical composition is formulated for use as a magnetic resonance imaging contrast agent. The pharmaceutical composition may be formulated for parenteral administration. In some implementations, the pharmaceutical composition is an injectable composition, and the compound according to formula IB is dissolved or suspended in the pharmaceutically acceptable carrier.

Embodiments of the disclosed contrast agents may be suitable for diagnostic imaging, such as magnetic resonance imaging. A method for magnetic resonance imaging may include administering to a subject an effective amount of a pharmaceutical composition comprising a compound according to formula IB, and performing a magnetic resonance imaging procedure on the subject. In one example, GdDOTFA (shown below), when bound to human serum albumin, achieved theoretical maximum relaxivity of 110 mM⁻¹s⁻¹, and is believed to be the first chelate to do so.

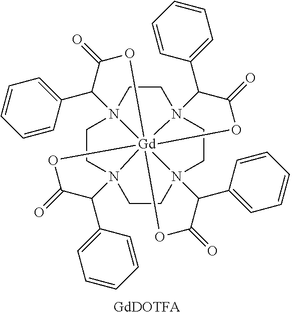

### III. STEREOSELECTIVE SYNTHESIS OF CONTRAST AGENTS

Embodiments of compounds according to formula IB may be stereoselectively synthesized. Advantageously, stereoselective synthesis is achieved using achiral starting materials. A brief overview of one exemplary synthesis is shown below:

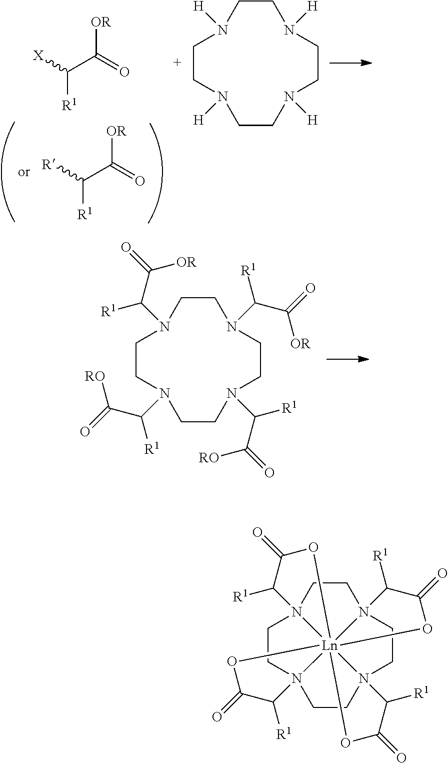

An alkylating agent,

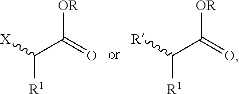

is prepared. R¹ is as previously described. In any of the foregoing or following embodiments, R¹ may be aryl, alkenyl, or alkynyl, and may be substituted or unsubstituted. R is a C₁-C₄ alkyl. In some implementations, R is methyl, ethyl, or isopropyl. In the alkylating agent

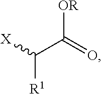

X is halo, e.g., Br, C1, or I. In certain implementations, X is Br. When R¹ is alkenyl, an alkylating agent, e.g.,

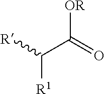

where R′ is a triflate, tosylate, or mesylate group, may be utilized in place of the halogenated starting material, with the remainder of synthesis proceeding as shown above.

In one embodiment, a glycine,

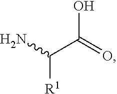

is halogenated to provide the corresponding halogenated glycine,

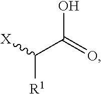

where X is Br, Cl, or I. The halogenated glycine is esterified to provide the alkylating agent,

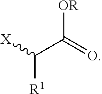

In some examples, the glycine is combined with a halide salt (e.g., NaBr, NaCl, or NaI) in a hydracid (HBr, HCl, or HI) and cooled to 0 °C. Sodium nitrite is gradually added, the solution is warmed to room temperature (e.g., 20-25 °C), and stirred until halogenation is complete. A base (e.g., KOH) is added to provide an alkaline pH, and the reaction mixture is extracted with diethyl ether. The aqueous layer is acidified and extraction is repeated. The combined extracts are dried and the solvents removed to provide the halogenated glycine. In some examples, a Fisher esterification is used to esterify the halogenated glycine. For example, the halogenated glycine may be dissolved in acidified methanol and heated with stirring until the halogenated glycine is esterified to form the alkylating agent. A base (e.g., K₂CO₃) is added to provide an alkaline pH, and the reaction mixture is extracted with diethyl ether. The extracts are dried and the solvents removed to provide the alkylating agent.

In an independent embodiment, a carboxylic acid,

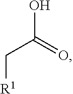

is esterified to provide

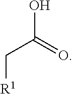

The esterified compound is then halogenated to provide the alkylating agent,

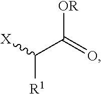

### When R¹ is

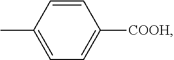

esterification may provide a diester

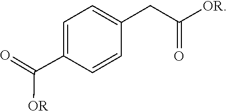

In some embodiments, the carboxymethyl compound is dissolved in a C₁-C₄ alkanol, acidified, and reacted under effective conditions to provide an esterified compound

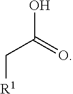

In some examples, the reaction proceeds under reflux with stirring for 12-24 hours, such as for 18 hours. The solvents are removed, and the residue is purified by extraction with water and diethyl ether. The esterified compound is recovered from the organic extract. In some aspects, the esterified compound is brominated by reaction with N-bromosuccinimide in acetonitrile to provide the alkylating agent,

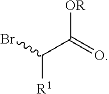

Alternatively, the esterified compound may be reacted with N-chlorosuccinimide or N-iodosuccinimide to provide

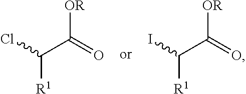

respectively. In one example, the reaction proceeds under ultraviolet irradiation (72-watt UV, 365-395 nm) with heating at 50 °C to 70 °C for 3-5 days, such as at 60 °C for four days. The solvent is removed. The residue is dissolved (e.g., in diethyl ether), filtered, washed, and purified, such as by column chromatography.

In some embodiments, the alkylating agent

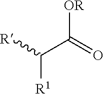

is prepared by hydroxylating an esterified alkene (e.g., with monoperoxyphthalate, MMPP) to introduce a hydroxyl group and then converting the hydroxyl group to a triflate:

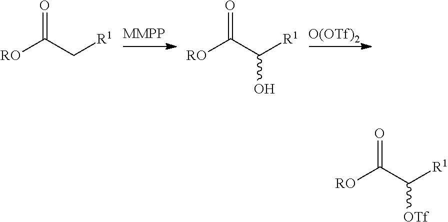

In other implementations, the esterified alkene is reacted to introduce a hydroxyl group as shown, and the hydroxyl group is then converted to a tosylate or mesylate group.

The alkylating agent,

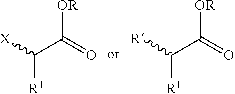

(e.g., when R¹ is alkenyl), is reacted with cyclen,

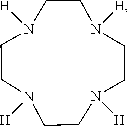

to provide an alkylated cyclen

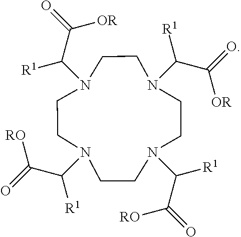

In some embodiments, the alkylating agent is reacted with the cyclen in acetonitrile and cesium chloride under effective conditions to provide the alkylated cycle. The effective conditions may include a temperature of from 50 °C to 70 °C and a reaction time, with stirring, of from 18-30 hours, such as 20-25 hours. Following the reaction, the solvents are removed. The residue is dissolved (e.g., in dichloromethane), and washed with water or a carbonate solution (e.g., K₂CO₃, pH 12-14). The aqueous layer is extracted (e.g., with dichloromethane). The organic layers are combined and dried, and the solvents are removed. The residue is purified, e.g., by column chromatography) to provide an alkylated cyclen.

The alkylated cyclen is saponified to remove R and provide a compound according to formula IA,

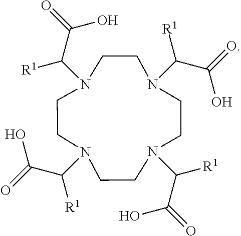

Saponification may be performed by combining the alkylated cyclen in an organic solvent (e.g., tetrahydrofuran) with an aqueous base, such as NaOH or KOH, under conditions effective to remove R. In some examples, saponification is performed at 50 °C to 70 °C with stirring for 12-60 hours, such as 16-20 hours or 45-50 hours. In some examples, the organic solvent is removed by evaporation, and the water is removed (e.g., by lyophilization) to yield the compound according to formula IA. The reaction mixture may be acidified (e.g., to pH 3) prior to water removal.

Finally, a lanthanide (Ln) salt, oxide, or triflate is chelated with the compound according to formula IA to provide a chelated compound according to Formula IB,

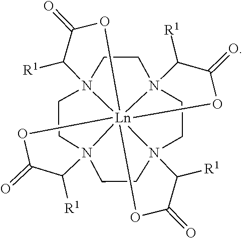

In some embodiments, Ln is Gd, Eu, or a combination thereof. In particular examples, Ln is Gd. Chelation is performed by combining the compound according to formula IA and the lanthanide salt, oxide, or triflate in aqueous solution. The reaction proceeds under effective conditions to provide the chelated compound according to Formula IB. In some examples, the effective conditions include a temperature 65 °C to 75 °C, with stirring for 36-96 hours. In one embodiment, the lanthanide salt is a lanthanide chloride, the pH is adjusted to be slightly acidic (e.g., pH 5-6), and the effective conditions include a temperature of 70 °C with stirring for 48 hours, followed by cooling and filtration. In an independent embodiment, the compound according to formula IA is combined with a lanthanide oxide, and the reaction is heated at 70 °C with stirring for 72 hours, followed by neutralization and filtration. In another independent embodiment, the compound according to formula IA is combined with a lanthanide triflate, the pH is adjusted to be slightly acidic (e.g., pH 5-6), and the effective conditions include a temperature of 70 °C with stirring for 48 hours, followed by cooling and filtration.

In some embodiments, the chelate according to formula IB comprises a diastereomer, and molecules of the diastereomer comprise a mixture of RRRR- and SSSS-enantiomers. In some implementations, at least 75%, at least 80%, at least 85%, at least 90%, or at least 95% of the compound molecules independently are RRRR- or SSSS-enantiomers.

### IV. EXAMPLES

Nomenclature: The complete systematic names of DOTA-derivatives are long. This leads to a tendency to employ acronyms that can more conveniently identify the compound of interest. DOTA itself is one such acronym: 1,4,7,10-TetraazacycloDOdecane-1,4,7,10-TetraAcetate. DOTMA was one of the first derivatives of DOTA to be reported. Its acronym denotes four acetate arms by the presence of the T (denoting tetra) and the A (denoting acetate. Note: A does not denote acetic acid: in the ligand the carboxylate is deprotonated). That these acetates are substituted is indicated by the M that separates the T and the A. Different substituents would be identified by a letter (or group of letters) denoting that substituent inserted between the T and the A. For instance, precedence would assign the letter P to a phosphonate groups and M indicates methyl. Table 1 provides a summary of the naming system.

TABLE 1

| Initial | Substituent | Acronym | Rationale | Other names |
|---|---|---|---|---|
| M | —Me | DOTMA | Precedence. | — |
| CE | 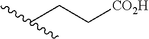 [SMILES: *CCC(=O)O] | DOTCEA | stands for Carboxy Ethylene. | TCE-DOTA and gDOTA |
| CP | 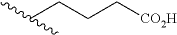 [SMILES: *CCCC(=O)O] | DOTCPA | stands for CarboxyPropylene. | aDOTA |
| AZ | 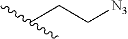 [SMILES: *CCN=[N+]=[N-]] | DOTAZA | identifies the AZide group well and precedence is consistent with the proposed system. | — |
| F | 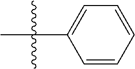 [SMILES: *c1ccccc1] | DOTFA | P is used for phosphonate but the one letter code for phenylalanine is F, on this basis F = phenyl. | — |
| B | 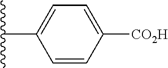 [SMILES: *c1ccc(C(=O)O)cc1] | DOTBA | stands for Benzoate. | — |
| NF | 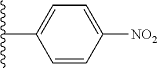 [SMILES: *c1ccc([N+](=O)[O-])cc1] | DOTNFA | stands for Nitrophenyl (F = phenyl). | |

Where four acetate arms are present but fewer than four substituents, a similar naming system is used where the number of each substituent is denoted by a subscripted numeral. For example, DOTF₃A has four acetate arms, three of which are substituted with phenyl groups. In this way multiple different substituents may be noted. For instance, DOTM₃B₁A would have four acetate arms, three of which are substituted with methyl groups with the fourth substituted by a benzoate.

Fewer than four acetate arms are denoted by numerals in place of the T and substituents can be denoted as laid out about, e.g., DO3MA (three acetate arms each with a methyl substituent), DO2F₁A (two acetate arms, one of which has a phenyl substituent). Finally, substituents could be (and in some cases have been) denoted prior to the “DOTA” acronym. Examples include TCE-DOTA and NB-DOTA, the former denoting four α-carboxyethyl groups and the latter denoting a single nitrobenzyl substituent on a carbon of the macrocycle. Given the possible confusion arising from the double usage of this notation, it is reserved for substituents located on a carbon of the macrocyclic ring.

Materials and Methods: All chemicals were purchased from commercial sources and used as received unless otherwise noted. Water refers to deionized water with a specific impedance >18 MΩ. Preparative HPLC was performed on a Waters™ 2545 system (Waters Corporation, Milford, MA) equipped with a 250× 50 mm Phenomenex® Luna C18 (2) column (Phenomenex, Torrance, CA). Chelates of both DOTFA and DOTBA were purified by eluting with water (0.037% w/w HCl) for 5 minutes followed by a linear gradient to 80% acetonitrile at 15 minutes with a flow rate of 50 mLmin⁻¹. The eluent was maintained at 20% water and 80% acetonitrile for a further 7 minutes. In all cases absorbance was monitored at 205 and 254 nm. ¹H and ¹³C NMR spectra were acquired on a Bruker® Avance® IIa spectrometer (Bruker Corporation, Billerica, MA) operating at either 400.13 and 100.61 MHz, respectively, or a Bruker® Avance® III NMR spectrometer operating at 600.17 MHz and 150.93 MHz, respectively. Melting points were determined on a Bibby Scientific SMP10 (Bibby Scientific Ltd, Stone, UK). The rotation of plane polarized light was measured on a Rudolph Research Analytical Autopol® 1 (Rudolph Research Analytical, Hackettstown, NJ). Infrared spectra we measured on a ThermoScientific Nicolet® iS10 FTIR spectrometer (Thermo Fisher Scientific, Inc., Waltham, MA) equipped with a total attenuated reflectance sample holder. Mass spectra were measured on a ThermoScientific LTQ-Orbitrap® Discovery mass spectrometer equipped with an Accula® autosampler (Thermo Fisher Scientific, Inc., Waltham, MA).

### Example 1

### Synthesis and Characterization of Tetraalkyl-α-Substituted DOTA Derivatives

The preparation of DOTMA is reported by various enantioselective syntheses starting from commercially available enantiopure S-hydroxy- or S-halo-propionates (Brittain, et al., Inorg. Chem. 1984, 23:4459-4466; Kang et al., Inorg. Chem. 1993, 32:2912-2918; Aime et al., Inorg. Chem. 2011, 50:7955-7965). By convention, the acronym DOTMA refers to the RRRR-enantiomer (Aime et al., Inorg. Chem. 2011, 50:7955-7965). The stereospecificity of each synthetic approach to DOTMA has not been tested—some purification steps (especially crystallization) may remove traces of unwanted diastereoisomers. Nonetheless, diastereoisomerically pure chelates can be readily obtained.

To establish a baseline for diastereoisomeric distribution in α-substituted DOTA derivatives, ±DOTMA was prepared using racemic 2-bromo propionic acid as shown in Scheme 1. Reagents and Conditions: i. t-butanol/cat. H₂SO₄/MgSO₄; ii. cyclen/Cs₂CO₃/MeCN; iii. TFA/40 °C; iv. EuCl₃/H₂O/pH 5.5. Carboxylates were protected as t-butyl esters and although the tetra-t-butyl protected ligand was subjected to column purification, this technique was not thought to have removed of one or more diastereoisomer from the mixture. The t-butyl esters were removed in trifluoroacetic acid and Eu³⁺ introduced under standard conditions (60 °C, pH 5.5).

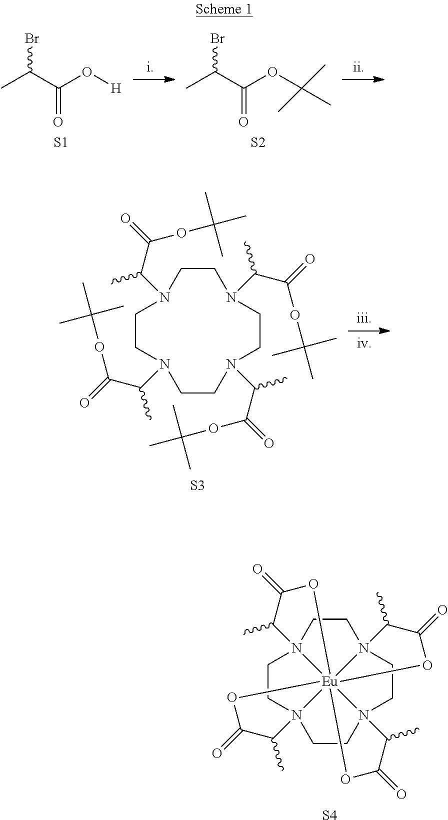

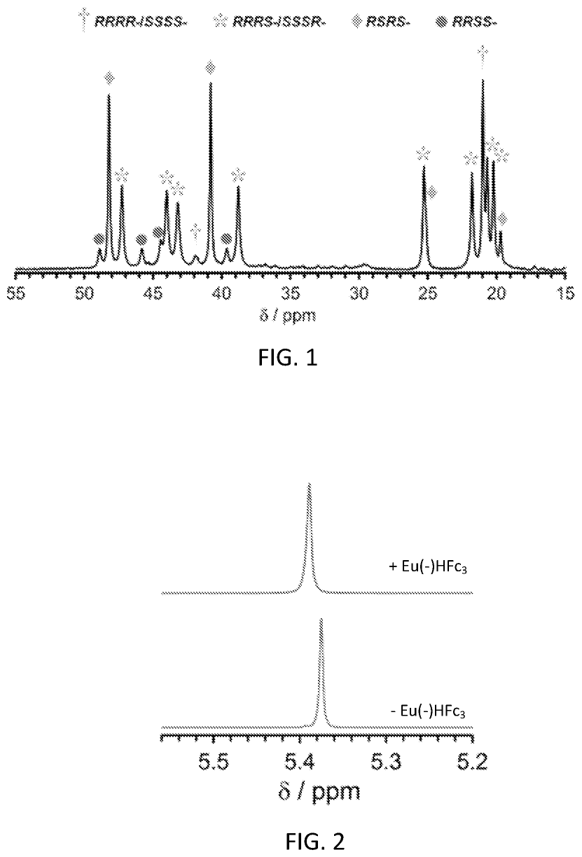

The NMR spectrum of ±EuDOTMA is shown in FIG. 1. Each diastereoisomer was identified by its symmetry and the observed SAP/TSAP ratio—comparing with EuDOTCEA (Howard et al., Chem. Commun. 1998, 1381-1382). (SAP=square antiprism, TSAP=twisted square antiprism.) The amount of each isomer present was determined by a line fitting analysis using the NUTS software package from Acorn NMR (Livermore, CA).

Comparing the distribution of diastereoisomers observed experimentally for #EuDOTMA with that predicted statistically (Table 2) reveals that the RRSS-isomer was produced in significantly lower quantities than expected.

TABLE 2

| | Isomer Distribution | | |
|---|---|---|---|
| Isomer | Statistical | ±EuDOTMA | Variance |
| RRRR-/SSSS- | 12.5% | 12.8% | +0.3% |
| RRRS-/SSSR- | 50.0% | 51.3% | +1.3% |
| RSRS- | 12.5% | 29.5% | +17.0% |
| RRSS- | 25.0% | 6.4% | −18.6% |

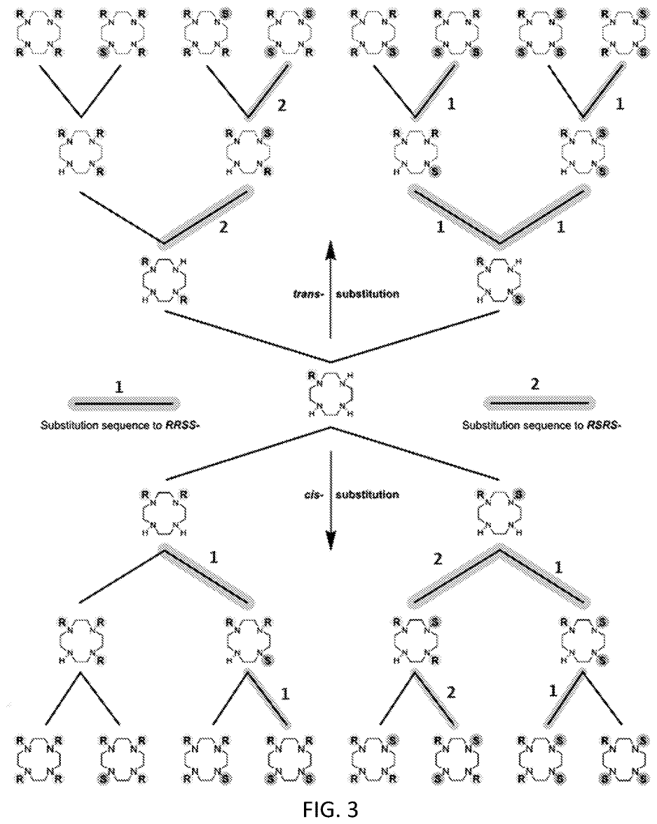

FIG. 3 shows the substitution sequences that lead to each isomer when the first alkylation step has afforded an R-configuration. The mirror image of these pathways exists for when the first alkylation yields an S-configuration. In FIG. 3, “R” and “S” are used to denote the absolute stereochemistry at the α-position of a pendant arm substituent. Substitutions resulting in an R-configuration are shown proceeding to the left, those resulting in an S-configuration are shown proceeding to the right. There are four pathways can potentially produce the RRSS-isomer, of these one is in competition with the RSRS-isomer the other three are in competition with the RRRS-/SSSR-isomer. But the amount of RRRS-/SSSR-isomer obtained is only slightly higher than the statistically predicted amount. The difference is made up by a larger than expected amount of the RSRS-isomer. But only two pathways afford the RSRS-isomer. If over-production of RSRS-arises primarily through a sequence involving trans-R,R-(upper left pathway), a concomitant decrease the production of both RRRR-/SSSS- and RRRS-/SSSR-would be expected. But this is not observed.

This implies that the sequences through cis-substitution (lower right pathway) that directly competes with RRSS-production must play a significant role in the distribution of isomers. There is a non-negligible contribution from sequences that follow from cis-substitution of cylen (Kruper et al., J. Org. Chem. 1993, 58:3869; 3867; Li et al., J. Org. Chem. 2003, 68:2956-2959). Since each alkylation reaction is functionally irreversible the isomeric distribution is controlled by kinetic factors; each substitution sequence is presumably associated with its own individual series of energy barriers. The observed isomeric distribution is the aggregate of the effects of these energy barriers. The interplay of these energy barriers is clearly complex, nonetheless it is possible to conclude that the overall energy barriers to each isomer follow the trend: RSRS-<RRRR-/SSSS-<RRRS/SSSR-<<RRSS-.

The ligands DOTFA and DOTBA were identified as targets of interest. Scheme 2 shows the synthetic route for the preparation of an enantiomerically pure alkylating agent to introduce phenyl groups into the α-position of DOTA (top). A general synthetic route for the preparation of DOTFA and DOTBA (bottom). Reagents and conditions: i. NaNO₂/HBr/KBr; ii. MeOH/H₂SO₄; iii. R-2, ±2 or 4/Cs₂CO₃/MeCN/60 °C; iv. KOH followed by HCl (pH 3). On the basis that stereochemistry must be controlled in the x-positions to avoid a mixture of diastereoisomeric chelates the synthesis of DOTFA was envisioned starting from commercially available R-phenyl glycine. The chiral α-amino acid was converted to the corresponding bromide R-1 with complete retention of stereochemistry by diazotization of the amine with nitrous acid in the presence of potassium bromide (Harfenist, J. Org. Chem. 1985, 50:1356-1359). Fisher esterification of R-1 in methanol afforded the suitably protected enantio-pure alkylating agent R-2 (FIG. 2). R-2 was used to tetra-alkylate cyclen in acetonitrile with caesium carbonate at 60 °C. To permit the evaluation of the stereochemical selectivity of this synthesis the esters were removed by saponification without performing extensive purification. The rare earth chelates of DOTFA were prepared in aqueous solution at pH 5.5 at 70 °C from the corresponding chloride or from oxide without adjustment of the pH.

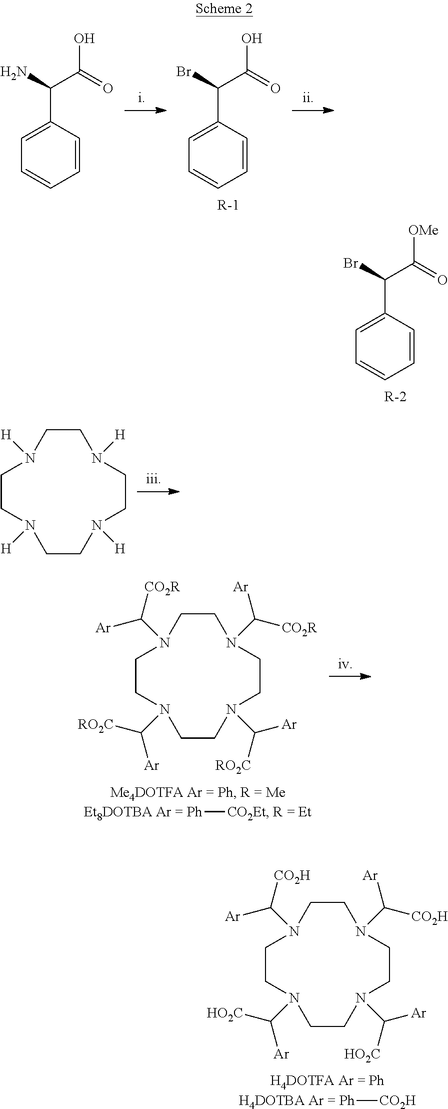

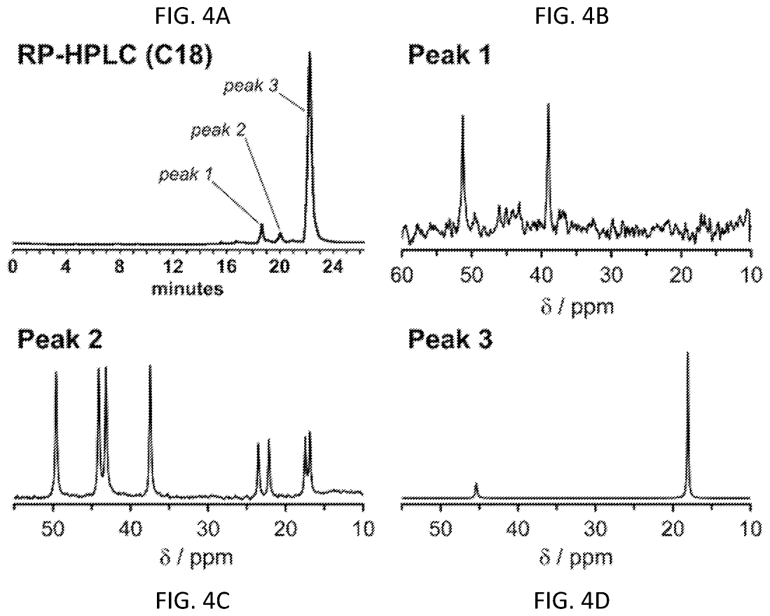

Purification of EuDOTFA was undertaken by RP-HPLC on a C18 column, the chromatogram of which is shown in FIG. 4A. The synthesis was found to produce one primary product (>90%), consistent with the strategy of controlling stereochemistry at each α-centre. Two smaller peaks with only slightly shorter retention times were observed in the HPLC trace. After separation by RP-HPLC chromatography, each peak was analyzed by ¹H NMR (FIGS. 4B-4D). The major reaction product (peak 3) exhibits C4-symmetry and was thus assigned to the expected SSSS-isomer. Peak 1 is found to possess C₂-symmetry and was assigned as the RSRS-isomer, peak 2 exhibits no symmetry and, based on the isomeric distribution observed for ±EuDOTMA, was assigned to the SSSR-isomer. The alkylating agent R-2 was confirmed to be enantiopure (FIG. 2) and so the production of unintended diastereoisomers may be attributable to a small degree of racemization during the alkylation reaction.

The synthesis of DOTFA chelates employed enantio-pure starting materials in procedures comparable to those used to obtain single enantiomers of other similar chelates (Kang et al., Inorg. Chem. 1993, 32:2912-2918; Aime et al., Inorg. Chem. 2011, 50:7955-7965; Kriemen et al., Eur. J. Inorg. Chem. 2015, 2015:5368-5378; Kriemen et al., Chem.—Asian J. 2014, 9:2197-2204). It was somewhat surprising therefore that these chelates exhibited effectively no rotation of plane polarized light (GdDOTFA: α_{D}²⁹⁶=+0.01 (water)). If racemization had occurred during the reaction, then a mixture of stereoisomers, comparable to that found in Table 2, would be expected. But the HPLC and NMR analyses unambiguously demonstrate that the RRRR-/SSSS-diastereoisomer was the predominant reaction product. Without wishing to be bound by a particular theory, these seemingly contradictory observations may arise because the ligand H₄DOTFA was prepared as a single enantiomer (SSSS-). But during chelation, inversion of the α-chiral center occurs easily. This will initially scramble the configuration of stereocenters, but if inversion is fast enough then eventually the mixture of stereoisomers deracemize to the thermodynamically most stable. The formation of an enol or enolate at the c-carbon has been demonstrated under either acidic (DOTCEA) (Woods et al., J. Am. Chem. Soc. 2000, 122:981-9792) or basic (DOTA) (Dickins et al., Chem. Commun. 1996, 697-698) conditions for certain chelates. DOTCEA chelates can deracemize but only under strongly acidic conditions (Woods, “Chiral Gadolinium Complexes as Potential Contrast Agents,” PhD thesis, Durham University, 1998). Under milder conditions each of the other isomers can be prepared (Howard et al., Chem. Commun. 1998, 1381-1832). Notably methyl substituted chelates do not appear to enolize (Tircso et al., Inorg. Chem. 2011, 50:7966-7979).

DOTFA is distinguished from other tetra-α-substituted chelates by its aryl substituents, which can potentially stabilize enolate intermediate facilitating deracemization under mild conditions. During the subsequent reprotonation step, bulky α-substituents will tend to drive the orientation of the pendant arms into the lowest energy diastereoisomer: RRRR-/SSSS-. Because this is a thermodynamically driven process the preferred isomer is not expected to be the same as that in the study of #EuDOTMA.

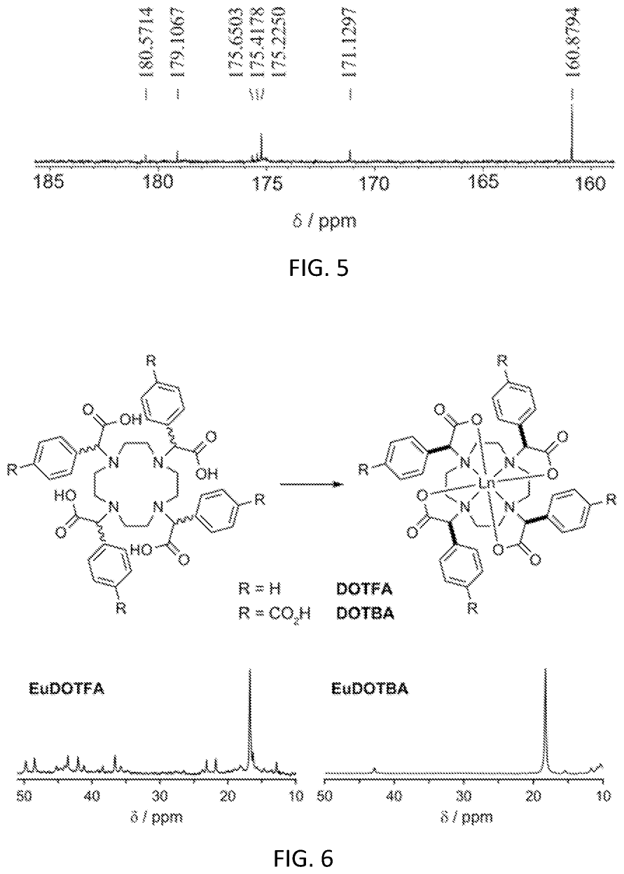

The ligands of DOTFA and DOTBA were prepared in racemic form. A suitable alkylating agent to produce DOTBA was prepared from an achiral source. 4-(Carboxymethyl)benzoic acid was protected as a diethyl ester also by Fisher esterification to afford 3 (Scheme 3). The benzylic carbon was then brominated with NBS in MeCN at 60 °C with constant irradiation at 365 nm-395 nm to afford ±4 after column chromatography. Cyclen was then exhaustively alkylated using either ±2 or ±4 in acetonitrile with caesium carbonate at 60 °C (Scheme 2). Care was taken in any purification steps to not inadvertently remove any diastereoisomer from the mixture—the presence of multiple isomeric chelates in the preparation of DOTBA was evidenced in the carbonyl region of the 13C NMR spectrum (FIG. 5). Esters were removed by saponification and the Eu³⁺ and Gd³⁺ chelates prepared from the chloride salts.

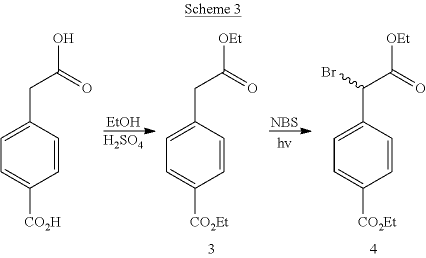

Although chelation of DOTFA was found to occur in the pH range commonly employed in this type of reaction (pH 5-6), chelation with DOTBA in this range was unsuccessful and only when a pH ≥12 was employed with heating could the metal ion be introduced into the ligand. NMR analysis of each reaction reveals that, in each case, the RRRR-/SSSS-isomer was the predominant reaction product (FIG. 6). Non-negligible amounts of other isomers were produced in the case of EuDOTFA produced from ±2, the distribution of isomers being comparable to that produced by using R-2. In contrast, only trace quantities of other diastereoisomeric chelates were discerned in the baseline of the spectrum of EuDOTBA. Appreciable quantities of another chelate could be observed in the up-field region of the spectrum although this does not correspond to a fully formed DOTA-type chelate. This chelate is attributed to residual Type I intermediate (Kálmán et al., Inorg. Chem. 2007, 46:5260-5270).

The observed distributions of isomers in these aryl-substituted chelates are important because they demonstrate that a single diastereoisomeric LnDOTA-type chelate can be prepared from an achiral starting material. This greatly expands the scope of the substituents that can be introduced into the α-position of DOTA-type chelates.

### Mechanism of Deracemization

RRRR-/SSSS- is the most thermodynamically stable isomer since this is the only isomer with congruence on all four pendant arms between the configuration at carbon and the helicity of the pendant arms. Racemization at the α-carbon leads to the conversion of the other isomeric chelates to the most thermodynamically stable isomer. What is more difficult to understand is why SSSS-H₄DOTFA should convert into RRRR-EuDOTFA. To do so the chelate must pass through two other isomers both of which are higher in energy than the starting chelate.

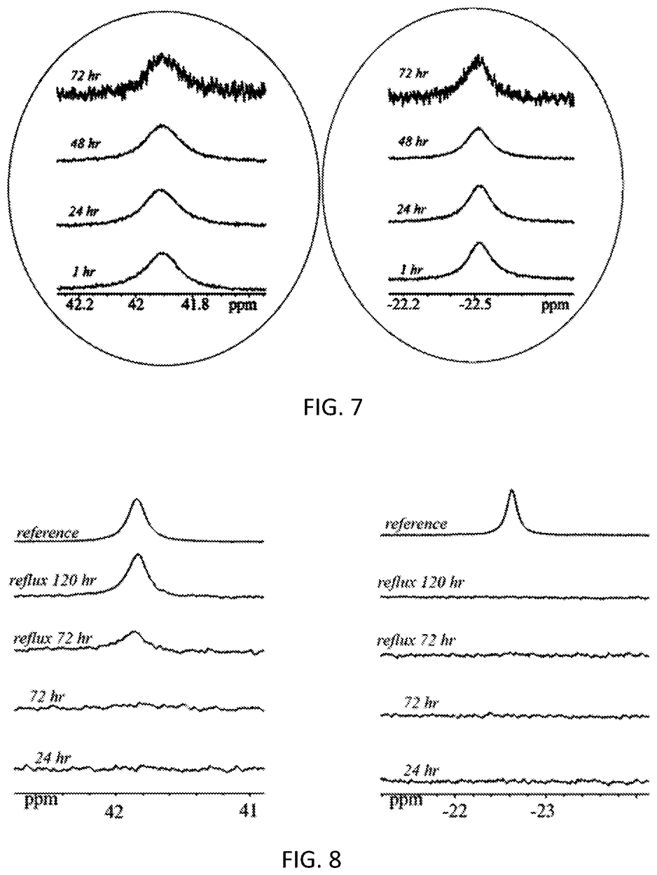

The α-protons of LnDOTA (Ln=Eu, Tb & Yb) can be exchanged for deuterium by heating (70 °C) in D₂O at pD 11.5 for 24 hours (Dickins et al., Chem. Commun. 1996, 697-698). The mechanism of this H/D exchange—formation of an enolate at the α-carbon—is the same as that expected in deracemization. To test whether deracemization occurs in the fully formed chelate, a sample of HPLC purified RRRR-/SSSS-Na₅EuDOTBA was incubated in a 1 mM solution of D₂O (pD 11) in a sealed NMR tube. Even when heating at 60 °C was extended to 72 hours, no significant decrease in the intensity of the α-proton resonance could be detected by ¹H NMR (FIG. 7). Table 4 shows the relative intensities of the SAP ¹H resonances of EuDOTBA corresponding to the most shifted axial proton (ax^{S}) and the chiral-proton. Intensities are normalized to the ax^{S}.). This compares with 90% incorporation for DOTA chelates (Ibid.). The absence of deuterium incorporation indicates that enolate formation in the fully formed chelate EuDOTBA does not occur to an appreciable extent under these conditions.

TABLE 4

relative ¹H intensity

| Time/hours | ax^{S} | α-CH |
|---|---|---|
| 1 | 1.00 | 0.96 |
| 24 | 1.00 | 0.64 |
| 48 | 1.00 | 0.97 |
| 72 | 1.00 | 1.00 |

Until the metal ion is introduced into the system there is no driving force for deracemization to the RRRR-/SSSS-isomer. If the fully formed chelate does not enolize, then presumably deracemization occurs during the chelate formation. The mechanism of chelation by DOTA is a two-step process. In the first step the metal ion associates with the carboxylate groups of DOTA to form, a so-called “Type I” intermediate complex (FIG. 7) (Toth et al., Inorg. Chem. 1994, 33:4070-4076; Stenson et al., Dalton Trans 2006, 3291-3293). In the Type I intermediate the metal ion interacts only with the pendant arms and the cyclen ring is diprotonated. In the second step, the rate determining step, the protons are removed from the cyclen ring, allowing the metal ion to drop down into the coordination cage of the ligand forming the final chelate. Generally the second step occurs easily at moderate pH, but occasionally unusually high pHs are required to deprotonate the cyclen ring—this is most common when ligands contain potential ligating groups in peripheral positions (Kálmán et al., Inorg. Chem. 2007, 46:5260-5270). The requirement for a high pH when chelating DOTBA is perhaps not surprising in this context.

The pendant arms of Type I complexes appear to bind cooperatively (Stenson et al., Dalton Trans 2006, 3291-3293), and so the same relationship between helicity and stereochemical configuration is expected. This suggests that deracemization could occur in the Type I intermediate. A solution of the DOTBA and EuCl₃ in D₂O at pD 13 (NaOD) was incubated in a sealed NMR tube at 60 °C for 72 hours followed by 120 hours at 100 °C. Reaction progress was monitored periodically by ¹H NMR spectroscopy.

No change in the NMR spectrum was observed after 72 hours (FIG. 8) and so the temperature was raised to 100 °C with incubation for a further 120 hours. The ax^{S} proton of the RRRR-/SSSS-isomer (at ~42 ppm) was seen to grow in after the temperature was increased to 100 °C, illustrating formation of the chelate. No other isomers were observed. The acetate peak (at ~−23 ppm) did not grow in, demonstrating deuterium incorporation at the α-carbon. This shows that deracemization is the result of enolization. Analysis of the most up- and down-field regions of the ¹H NMR spectrum of FIG. 8 shows the formation of the RRRR-/SSSS-isomer as the axial ring proton (ax^{S}) resonance at 42 ppm grew in over time. But even as the chelate formed, no peak was observed in the most up-field region of the spectrum where the α-CH resonance was expected to appear. These results are consistent with incorporation of deuterium at the α-carbon. Since deuterium was not incorporated in the fully formed “Type II” chelate, it may be concluded that this occurs only in the Type I intermediate. Furthermore, despite the use of a racemic ligand sample, no other stereoisomeric chelates were observed in the reaction. It was concluded that deracemization occurs via enolate formation in the Type I intermediate during chelation. This may also explain the racemization of a single enantiomer of a ligand to a mixture of enantiomers in the chelate. The difference in energy between isomers may be less pronounced in the Type I complex than in the Type II complex. This may mean that the intermediates through which this conversion must pass are more accessible than they appear by considering the fully formed chelate.

To verify the involvement of an enolate in the mechanism of deracemization, preparation of a DOTA derivative substituted with both a phenyl and a methyl group on each pendant arm was attempted. By eliminating the α-proton, this substitution pattern would block the formation of an enolate and would therefore be expected to afford a mixture of diastereoisomer chelates despite the presence of an α-aryl substituent. Accordingly, ethyl 2-phenylpropanoate 5 was prepared from the corresponding acid prior to bromination of the benzylic positions using an identical procedure for that was used to prepare 4 (Scheme 4). Reagent and Conditions: i. EtOH/H₂SO₄/Δ; ii. cyclen/Cs₂CO₃/MeCN. However, when the disubstituted bromide 6 was used to alkylate cyclen under the same conditions used for DOTFA and DOTBA, exhaustive alkylation could not be achieved even after protracted reaction times (>4 weeks). Presumably steric effects limit the third and fourth alkylation reaction: analyzing reaction progress by mass spectrometry showed that the extent of tri-substitution of cyclen was low, with the reaction essentially stalling at di-substitution.

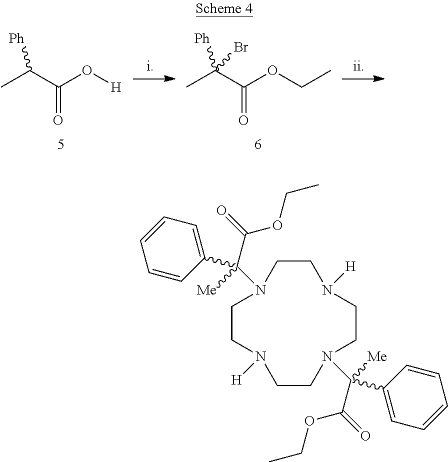

### Effect of Chelation Conditions on Isomeric Distribution

The divergence in isomeric distribution between EuDOTFA and EuDOTBA is notable. These two chelates were prepared under quite different pH regimes: the chelation of DOTBA was undertaken at significantly higher pH and exhibited almost complete deracemization. This suggests that higher reaction pHs promote enolate formation, which in turn leads to more effective deracemization. To evaluate the effect of pH on the extent of deracemization, Eu³⁺ was introduced to samples of DOTFA in buffered media from pH 4 to pH 10.

2.2 mM solutions of ±DOTFA were prepared in 50 mM acetate buffer (pH 4, 5 & 6) and ammonium acetate buffer (pH 7, 8 & 10). 3.0 equivalents of EuCl₃ were added and each solution was heated with stirring at 70 °C for 48 hours. Acetate buffers are considered volatile buffers, permitting removal of most of the buffer prior to NMR analysis. They are also known to be “coordinating” buffers, which may play a role in the kinetics of the chelation reaction. But given that acetate concentration is the same in each buffer, the results of these experiments can justifiably be compared with one another, although not necessarily with those of reactions with a different or no buffer. At the end of the reaction, each sample was lyophilized and redissolved in D₂O. The ¹H NMR spectrum of each was recorded with pre-saturation suppression of the residual acetate buffer peak. The amount of each isomer present was determined by the line-fitting procedure described above.

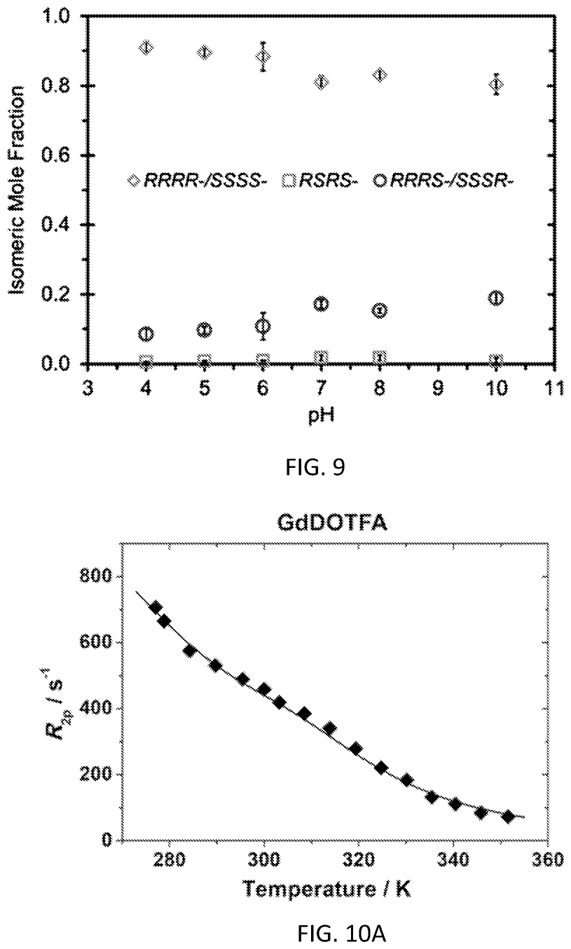

Under all pH regimes, the desired RRRR-/SSSS-isomer was the predominant reaction product. When the reaction was performed at the highest pH (10), this isomer made up 80% of the produced EuDOTFA. Unexpectedly, as the pH was reduced, the proportion of the RRRR-/SSSS-isomer increased, eventually rising to 91% when the reaction was performed at pH 4 (FIG. 9). Of the other isomers, only RSRS- and RRRS-/SSSR-were produced in measurable quantities. Only 1-2% of the RSRS-isomer was produced, the quantity of this isomers seemingly largely unaffected by pH. In contrast the amount of RRRS-/SSSR-isomer produced exhibited a clear decrease as the pH was increased.

The presence of a strongly Lewis acidic metal ion is important for the ability of the α-stereocenter to invert rapidly—the stereocenter of phenyl glycine is not found to be so labile to inversion. Without wishing to be bound by a particular theory, that inversion is neither an acid nor a base catalyzed process but is initiated by the proximate Lewis acid resulting in bidentate chelation by the carboxylate as shown in Scheme 5 where H₂EuL²⁺ is the Type I complex, H₂Eu(L-H)⁺ the Type I complex in which an enolate has formed, and EuL⁻ is the fully formed Type II complex.

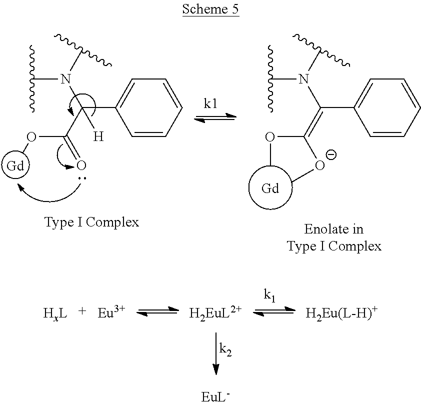

This would explain why inversion is found to occur during chelation when the Type I complex is present, but not once the Type II complex has formed. In the fully formed complex, the carboxylates are incapable of bidentate coordination. The rate determining step for inversion of the α-stereocenter in the Type I complex is the formation of the enolate (designated by the rate constant k₁). Conversion of the Type I complex to the Type II complex is the rate determining step of the chelation reaction (designated by the rate constant k₂) (Toth et al., Inorg. Chem. 1994, 33:4070-4076). This process involves removing two protons from the cyclen ring and is intrinsically pH dependent. Thus, as the pH is reduced the rate at which the Type II chelate is formed decreases, while the rate at which stereochemical inversion occurs may be unaffected. The faster rate of enolization relative to that of chelate formation would explain the greater proportion of RRRR-/SSSS-isomer obtained at lower pH.

The distribution of isomers appears to be related to relative rates arising from k₁ and k₂, this may afford an opportunity to exert control by changing the temperature. However, the isomeric distribution at pH 4 was not found to vary significantly over the temperature range 40 °C-70 °C. Table 5 shows the isomeric distribution as mole fractions determined for EuDOTFA chelates prepared from ±DOTFA at pH 4 at different temperatures. Reaction time=48 hours. This suggests that reaction conditions may be less important than structural consideration in determining isomeric distribution.

TABLE 5

| Isomer | 40 °C | 50 °C | 60 °C | 70 °C |
|---|---|---|---|---|
| RSRS- | 0.004 ± 0.002 | 0.009 ± 0.0004 | 0.010 ± 0.002 | 0.005 ± 0.001 |
| RRRS-/SSSR- | 0.101 ± 0.028 | 0.092 ± 0.043 | 0.112 ± 0.014 | 0.085 ± 0.016 |
| RRRR-/SSSS- | 0.895 ± 0.029 | 0.900 ± 0.043 | 0.878 ± 0.016 | 0.910 ± 0.017 |

The difference in isomeric distribution between EuDOTFA and EuDOTBA is larger than any change affected by change in conditions. The benzoate carboxylate is the only feature that distinguishes DOTBA from DOTFA. In FIG. 6, the NMR spectrum of EuDOTBA has peaks that were attributed to the residual Type I complex, this implies that the peripheral carboxylate may cause a decrease in k₂ by stabilizing the Type I intermediate (Kálmán et al., Inorg. Chem. 2007, 46:5260-5270). This would increase the lifetime of the intermediate, affording more time for the inversion of stereochemistry to occur and possibly accounting for the almost complete deracemization of EuDOTBA.

The chemistry of tetra x-substituted DOTA derivatives has previously been limited by the need to consider stereochemistry during synthesis and the availability of suitable enantiopure starting materials. However, the foregoing results have shown that these restrictions no longer apply when the substituents are aryl groups. Achiral starting materials can be used to generate racemic α-carboxy alkylating agents, which produce substituted DOTA ligands as mixtures of diastereoisomers. During chelation the aryl substituents stabilize enolate formation at the α-position facilitating deracemization, under mild conditions, of the ultimate chelate. Alkyl substituents are not generally found to facilitate this type of deracemization. And in the rare instances where they do, strongly acidic chelation conditions are required. Deracemization occurs in the intermediate Type I complex and is thought to be initiated by bidentate ligation of the Lewis acid by the carboxyl. Formation of the final Type II complex competes with deracemization because bidentate ligation and thus enolization are not possible in this chelate. Reaction conditions that slow the rate of chelate formation (lower pH) were found to improve the extent of deracemization. But structural considerations are more important in governing the extent of deracemization than reaction conditions. Both aryl substituents investigated here afford >90% of the RRRR-/SSSS-isomer. These chelates were found to be freely soluble in water but crystallization from water can be used to remove the minor isomers.

### Example 2

### Relaxivity of α-Aryl Substituted GdDOTA

There is a need to reduce the amount of Gd³⁺ administered for an MRI exam while retaining its diagnostic efficacy. The emergence in the early 2000s of nephrogenic systemic fibrosis (NSF) in patients with compromised renal function who had undergone contrast-enhanced MRIs raised concerns over the safety of Gd³⁺ chelates (Cowper et al., Am. J. Dermatopathol. 2001, 23:383; Kuo et al., Radiology 2007, 242:647-649). The use of contrast agents in MRI is also causing pollution. Thus, there is a need for Gd³⁺ chelates that can be used at lower doses.

The Gd³⁺ chelates used as MRI contrast agents are notoriously inefficient, requiring high doses: typically 1.0-1.5 g per dose. The relaxivity of a chelate depends upon the number of solvent water molecules that can coordinated directly to the Gd³⁺ (q), the distance of their protons from the metal (r_{GdH}), the rate at which they exchange with the bulk solvent (1/τ_{M}) as well as the rate at which the chelate tumbles (1/TR) and the electronic relaxation parameters 42 and tv. The contrast agents currently in use are sub-optimal: exchanging water too slowly and tumbling too rapidly (Sherry et al., Curr. Opin. Chem. Biol. 2013, 17:167-174).

GdDOTFA and GdDOTBA were prepared as described above. The relaxometric analyses described herein were performed using previously described instrumentation and methods (Webber et al., Inorg. Chem. 2020, 59:9037-9046).

GdDOTA itself has sub-optimal water exchange kinetics (Aime et al., Inorg. Chem. 2011, 50:7955-7965). DOTA chelates are found to adopt both square antiprismatic (SAP) and twisted square antiprismatic (TSAP) coordination geometries. This is of significance because water exchange in TSAP isomers is found to be up to 100× faster than the SAP isomer (Woods et al., Angew. Chem. In. Ed. 2003, 5889-5892; Aime et al., Angew. Chem. Int. Ed. 1998, 37:2673-2675; Woods et al., J. Am. Chem. Soc. 200, 122:9781-9792). GdDOTA predominates as the SAP isomer, increasing the proportion of TSAP isomer is a strategy for improving water exchange kinetics (Woods et al., Angew. Chem. In. Ed. 2003, 5889-5892; Dai et al., Nat. Commun. 2018, 9:857).

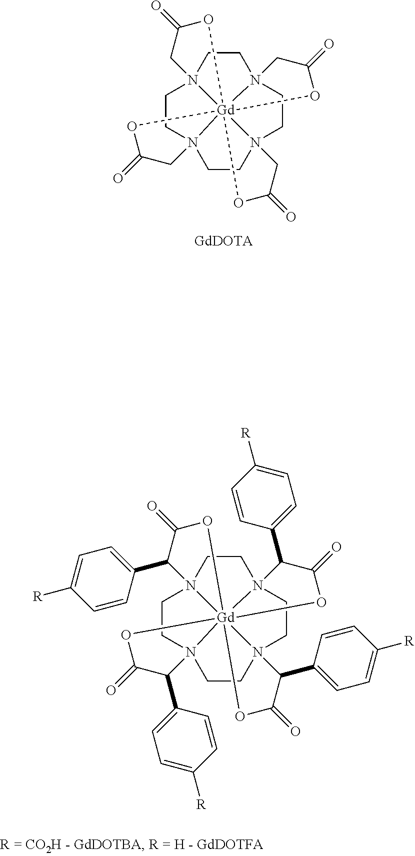

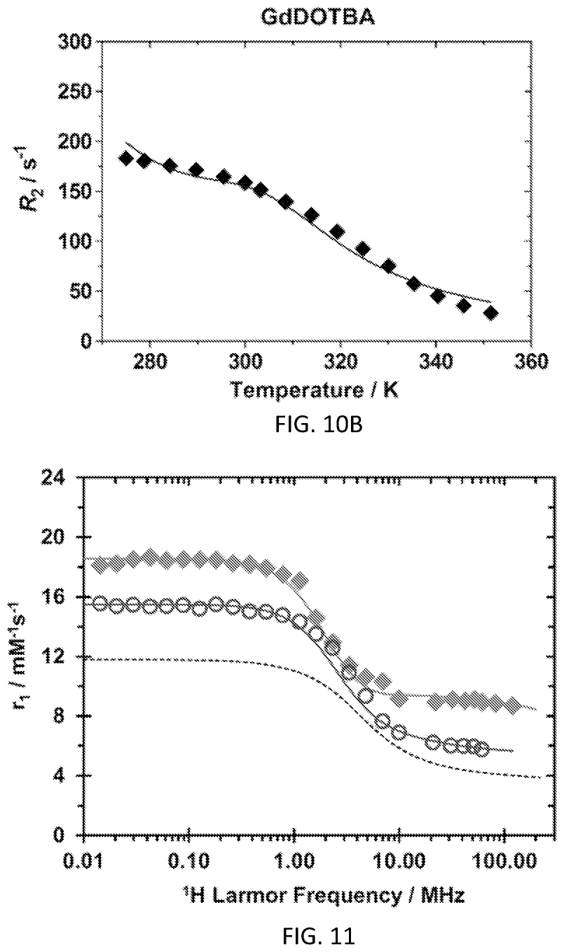

Substituting the α-position of the pendant arms of a DOTA chelate as shown above was found to increase the TSAP/SAP ratio (298 K) from 1:4 (EuDOTA) to 7:1 (EuDOTFA) and 10:1 (EuDOTBA). This change in isomeric ratio was found to have a profound effect on increasing the rate of water exchange. Analyzing the Gd³⁺ chelates by variable temperature 170 NMR afforded the water exchange lifetimes: τ_{M}=19 ns (GdDOTFA) and τ_{M}=10 ns (GdDOTBA) (FIGS. 10A-10B). These aryl substituted DOTA derivatives exhibit water exchange that are optimal for achieving the highest relaxivities.

Information about GdDOTFA and GdDOTBA can be obtained from a quantitative analysis of the nuclear magnetic relaxation dispersion (NMRD) profiles of these chelates which measure relaxivity as a function of the applied magnetic field (B₀). FIG. 11 shows the NMRD profiles at 298 K of both GdDOTFA (open circles) and GdDOTBA (closed diamonds). For reference, the fit of the ¹H NMRD profile of GdDOTA is shown (dashed line). The profiles are notable for several reasons. The relaxivity at all fields is significantly higher than that of GdDOTA, this can be attributed to the larger size of these chelates-slower rotational tumbling (longer τ_{R}). At low fields this difference is especially pronounced: indicative of more favourable electronic relaxation properties. This is confirmed by the results of simultaneously fitting the NMRD profiles with the VT ¹⁷O data (Table 6). Parameters fixed during fittings: r_{GdH}=3.0 Å, ²⁹⁸D=2.24×10⁵ cm² s⁻¹, a=4.0 Å, A_{O}/h=−3.6×10⁶ rad s⁻¹. The relationship between coordination chemistry and electronic relaxation is not entirely clear, but the aromatic substituents appear to shield the chelate from collision, which reduces the rate of modulation of the zero-field splitting (ZFS). Additionally, the square of the trace of the ZFS tensor (Δ²) is significantly smaller than in most DOTA type chelates. Together these have the potential to lift the limiting effect of electronic relaxation on relaxivity typically observed for DOTA type chelates.

TABLE 6

| Parameter | GdDOTFA | GdDOTBA | GdDOTA^{[a]} |
|---|---|---|---|
| ^{0.5}r₁²⁹⁸/mM⁻¹s⁻¹ | 8.1 | 11.4 | 4.8 |
| A_{O}/ℏ 10⁶ rads⁻¹ | −3.6 ± 0.1 | −3.6 ± 0.2 | −3.7 |
| t_{M}²⁹⁸/ns | 19 ± 0.6 | 10 ± 1 | 261 |
| t_{R}²⁹⁸/ps | 161 ± 10 | 274 ± 7 | 66 |
| Δ²/10¹⁸ s⁻² | 6.8 ± 0.2 | 10.0 ± 0.4 | 16 |
| t_{V}²⁹⁸/ps | 25 ± 2 | 31 ± 1 | 7.7 |

^{[a]}Sherry et al., Curr. Opin. Chem. Biol. 2013, 17: 167-174.

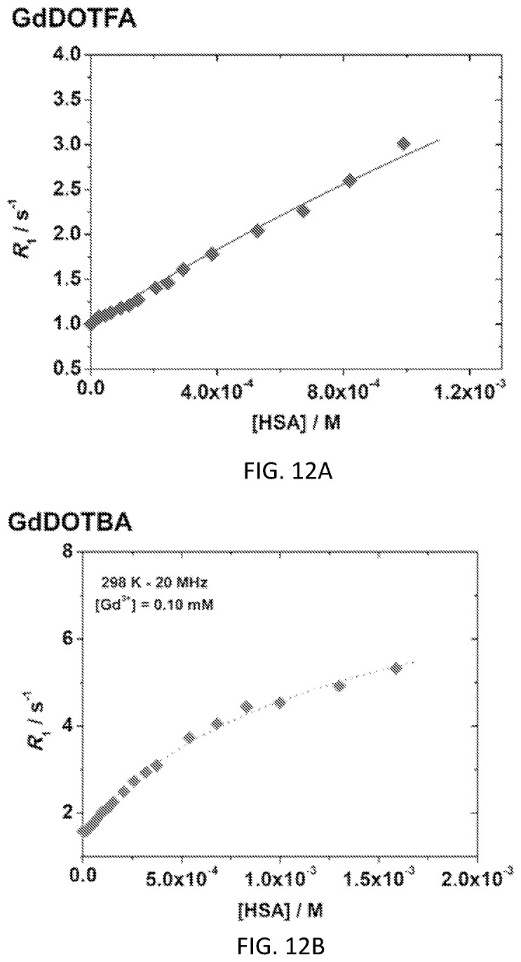

To achieve the highest relaxivities from a Gd³⁺ chelate it is necessary to make a substantial reduction in the rate of tumbling (Zhang et al., Angew. Chem. Int. Ed. 2003, 44:6766-6769). One commonly employed strategy for achieving this goal is binding to a macromolecule such as a protein. Human serum albumin (HSA) is a commonly used protein for this purpose because a simple hydrophobic interaction can be used to couple the motion of the chelate to that of the protein (Botta et al., Eur. J. Inorg. Chem. 2012, 2012:1945-1960). GdDOTFA and GdDOTBA both have aromatic substituents with the potential to bind to HSA. Accordingly, HSA was titrated into solutions of GdDOTFA (0.09 mM) and GdDOTBA (0.1 mM), and binding was monitored by relaxometry at 0.5 T and 298 K (FIGS. 12A and 12B). Fitting these data using a simple 1:1 binding model shows that the binding of both chelates to HSA was quite weak. Perhaps surprisingly, GdDOTBA binds to HSA more strongly (Kₐ=1143 M⁻¹) than GdDOTFA (Kₐ=220.1 M⁻¹). The two chelates differ primarily in the peripheral carboxylates of GdDOTBA; presumably these groups increase chelate hydrophilicity thereby decreasing the hydrophobic interactions between chelate and protein. Given the differences in binding constants, it may reasonably be supposed that the nature of the interaction with HSA is different for each of these two chelates. This may give rise to differences in other factors such as chelate orientation and freedom of the chelate to rotate locally, each of which may impact relaxivity (Avendano et al., Chem. Commun. 2007, 4726). The relaxivity of GdDOTBA was found to increase by 521% upon binding: the relaxivity of the chelate when bound to HSA: r₁^{bound}=59.4 mM⁻¹s⁻¹ at 20 MHz and 298 K. This significant improvement in relaxivity was modest in comparison to the 1,361% increase measured for HSA-bound GdDOTFA: r₁^{bound}=110.3 mM⁻¹s⁻¹ at 20 MHz and 298 K, even though the association between GdDOTFA and HSA is weak.

To the inventors' knowledge, this is the first discrete q=1 Gd³⁺ chelate to achieve theoretical maximal relaxivity. The calculation of maximal relaxivity stipulates that the chelate contains a single Gd³⁺ ion and has just one open binding site for coordination by water. So, while chelates with relaxivities as high as this have been reported (Ananta et al., Nat. Nanotechnol. 2010, 5:815-821; Courant et al., Angew. Chem. 2012, 124:9253-9256), they fall outside the scope of the calculation. Such high relaxivity suggests that DOTFA binds to HSA in a way that effectively couples the motion of the chelate to that of the protein, it also indicates the value of optimizing of several key parameters: τ_{M}, Δ² and τ_{V}.

Replacing the current crop of contrast agents with ones that can perform equally well at lower doses requires lower molecular weight chelates. Increasing the effective molecular mass of a Gd³⁺ chelate sufficiently to maximize relaxivity will cause an agent to extravasate and excrete more slowly. This reduces its utility as a contrast agent and potentially recreates the safety concerns of NSF. In terms of performance as a low molecular weight contrast agent, GdDOTBA may be useful.

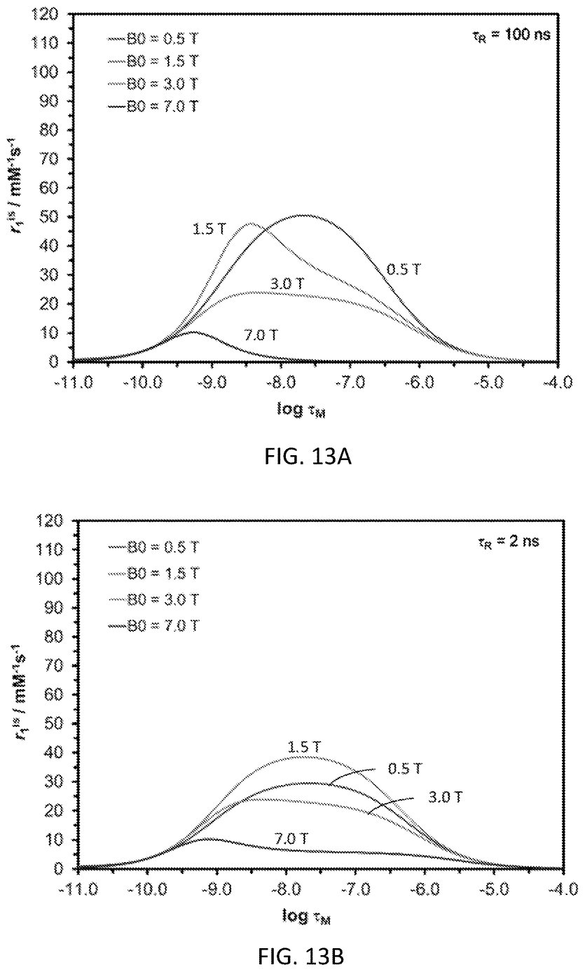

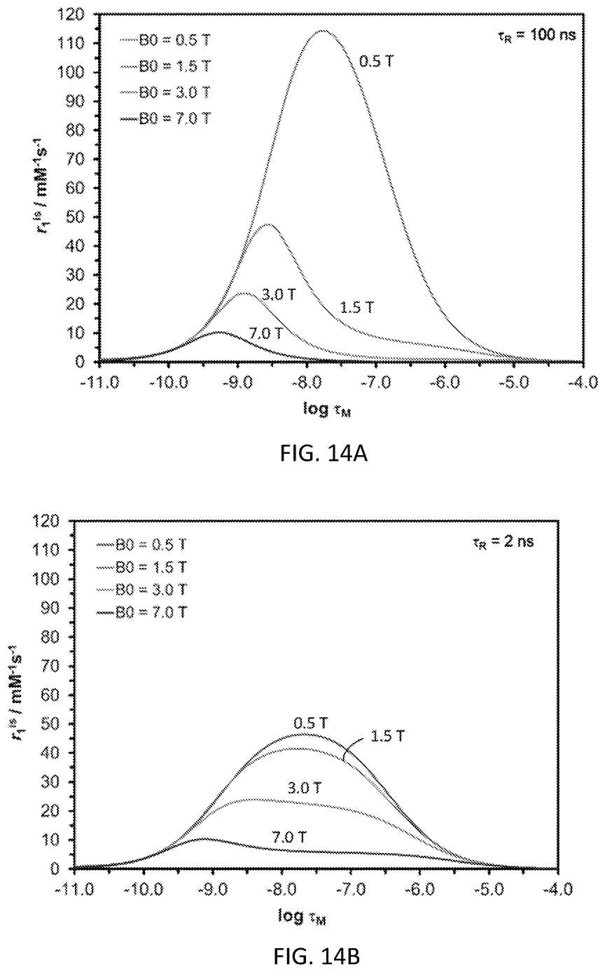

At higher B₀, GdDOTA exhibits the slow, steady decrease in relaxivity generally observed for Gd³⁺ chelates as the magnetic field increases. GdDOTFA exhibits a similar decrease. But in the NMRD profile of GdDOTBA there is a small relaxivity “hump” centered around 1 T; this is indicative of a more slowly tumbling chelate. Because GdDOTBA is still relatively small, this hump is small. Because water exchange in GdDOTBA is very fast, this hump is pushed to higher fields than usual (0.5 T is typical) (FIGS. 13A-13B and 14A-14B). The result of these considerations is that the relaxivity of GdDOTBA is both quite high and more or less unchanged over the range B₀=0.5-3.0 T. At 298 K the relaxivity at 3.0 Tis 11.35 mM⁻¹s⁻¹ which compares favorably with 11.7 mM⁻¹s⁻¹ at 1.5 T and 11.4 mM⁻¹s⁻¹ at 0.5 T.

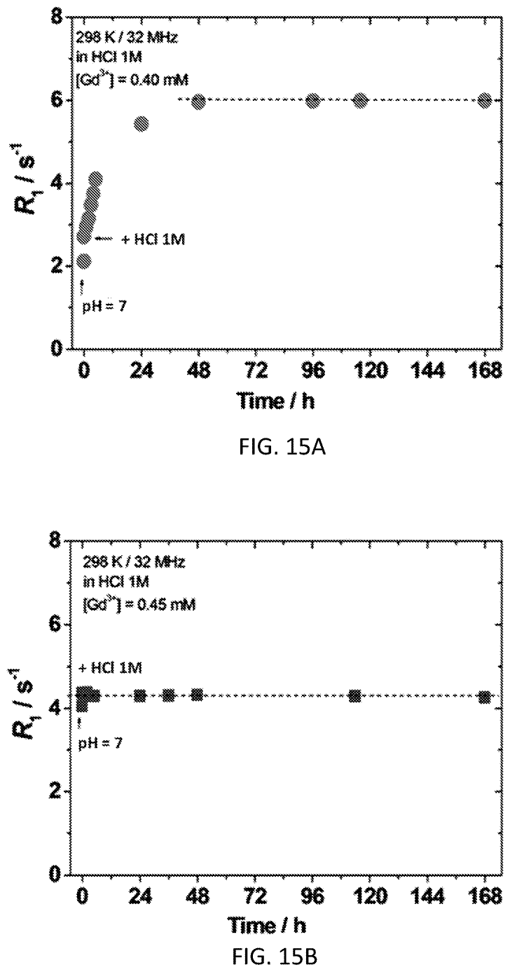

FIGS. 15A and 15B show the “most stable” currently available contrast agent GdDOTA (FIG. 15A) and GdDOTFA (FIG. 15B) incubated in 1 M HCl. As shown in FIG. 15A, R₁ goes up as the Gd ion is released from the chelate. No increase in R₁ is seen in FIG. 15B, indicating that even under the aggressive conditions of 1 M HCl, the Gd ion seems immune to dissociation. Even after several days, little or no Gd was released, suggesting that these chelates are likely to be much safer than any existing agent.

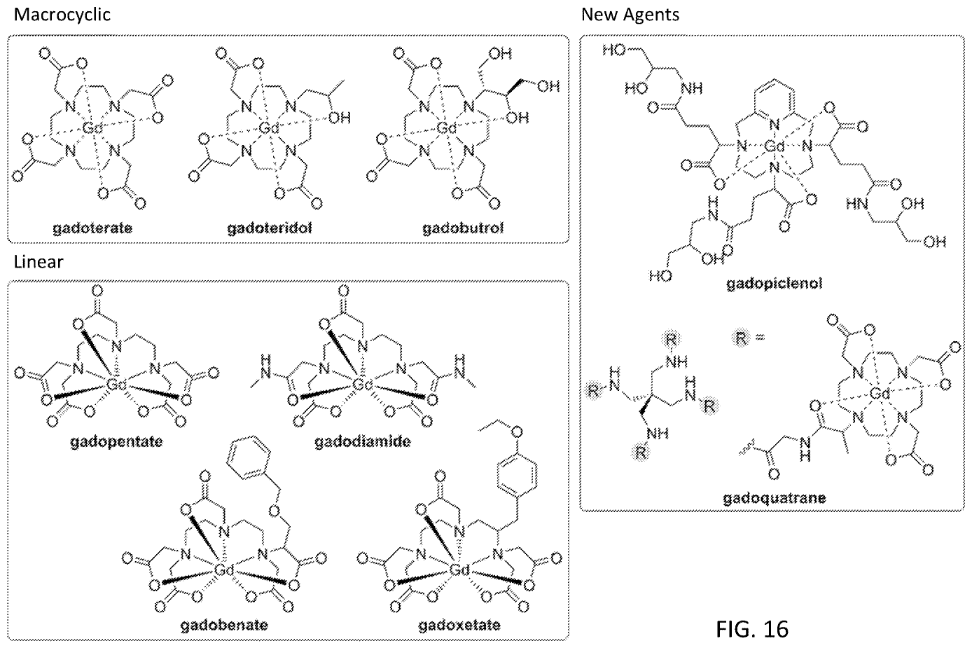

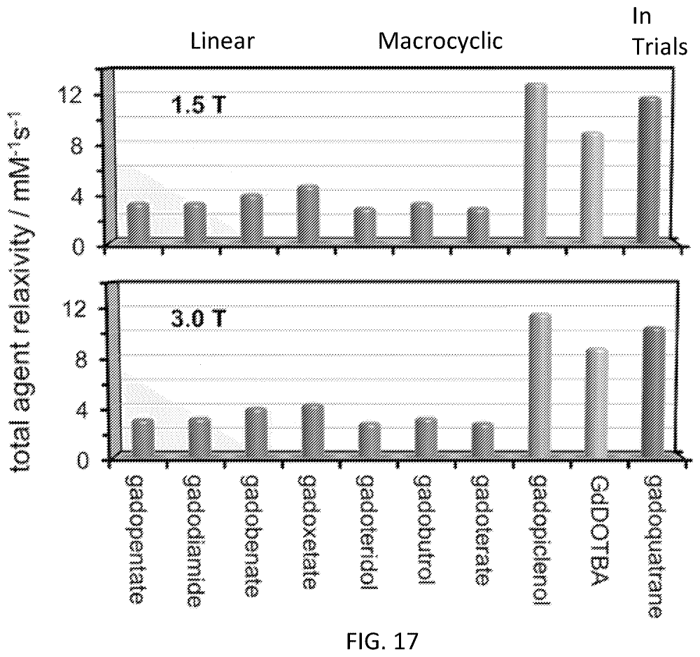

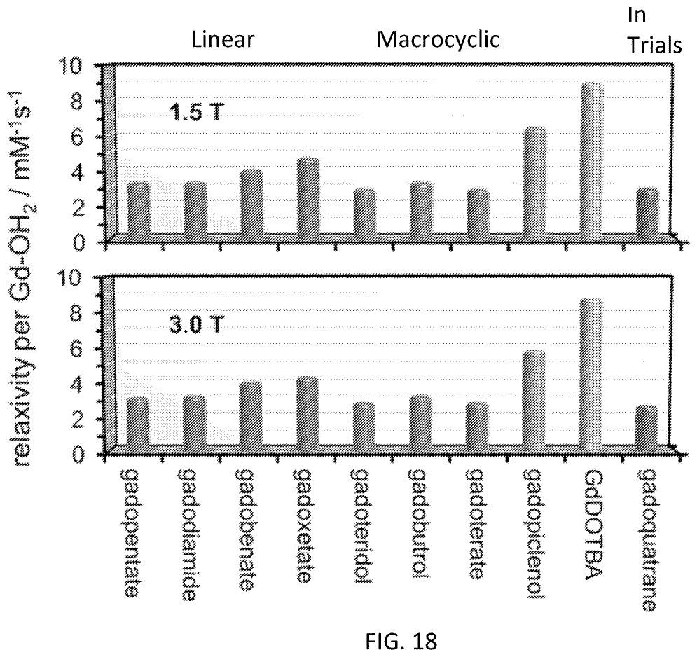

The relaxivity of GdDOTBA compares very favourably with established clinical contrast agents (Laurent et al., Contrast Media Mol Imaging 2006, 1:128-137), between 2 and 3 times higher (FIGS. 16-18). With respect to FIG. 16, coordinated water molecules are not shown, but these chelates are coordinatively saturated when CN-9, the maximum number of coordinated water molecules can therefore be calculated by subtracting the denticity of the ligand from 9. Only two more recent entries—gadopiclenol and gadoquatrane—have relaxivities (Robert et al., Radiology 2020, 294:117-126; Lohrke et al., Invest. Radiol. 10.1097/RLI.0000000000000889) even close to that of GdDOTBA at 310 K and either 1.5 or 3.0 T. However, care is required when comparing these two agents with GdDOTBA or any of the established clinical agents. Gadopiclenol employs a heptadenate ligand opening a second coordination site to water, a strategy that was once considered risky for a chelate that is to be used in vivo because of the risk of compromising chelate robustness. Gadoquatrane increases the number of Gd³⁺ ions from 1 to 4, effectively quadrupling the dose. Neither of these chelate meet the criteria of a Gd³⁺ chelate that has one site open for coordination by water. FIG. 18 shows relaxivity per Gd³⁺ coordinated water molecule-which provides a better assessment of the ability of the Gd³⁺ ion in each agent to relax solvent water protons (Livramento et al., Angew. Chem. Int. Ed. 2005, 44:1480-1482; Caravan et al., Angew. Chem. Int. Ed. 2007, 46:8171-8173). It is clear from FIG. 18 that GdDOTBA is a particularly effective relaxation agent with the potential to reduce dose by a factor of two or more, maintain a good safety profile and perform well at high fields.

In summary, introducing aryl groups into the x-position of the pendant arms of GdDOTA increases the proportion of TSAP isomer, which in turn accelerates the kinetics of water exchange. Without wishing to be bound by a particular theory, the α-aryl substituents create a symmetrical ligand field that is shielded from modulation by collision, improving the electronic relaxation parameters. They can also be used to bind to macromolecules and slow the tumbling of the chelate in solution. They have allowed, for the first time, simultaneous optimization of the parameters governing relaxivity. GdDOTFA, when bound to the HSA, is the first chelate to afford the peak relaxivity at 0.5 T and 298 K. GdDOTBA, as a discrete chelate, exhibits unprecedentedly high relaxivity even at the higher fields (1.5 T and 3.0 T) typically used in clinical MRI. These relaxivity gains are all achieved while preserving octadentate coordination of the DOTA framework—these chelates are expected to retain the robustness (and thus safety) associated with DOTA chelates.

### Example 3

### Additional Syntheses

Additional chelates have been prepared according to the synthesis described in Example 1, where Ar is

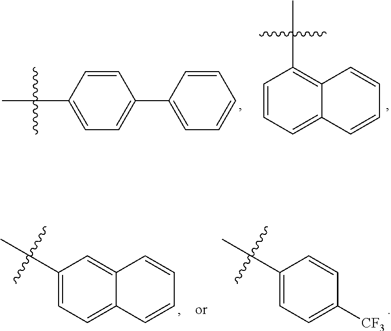

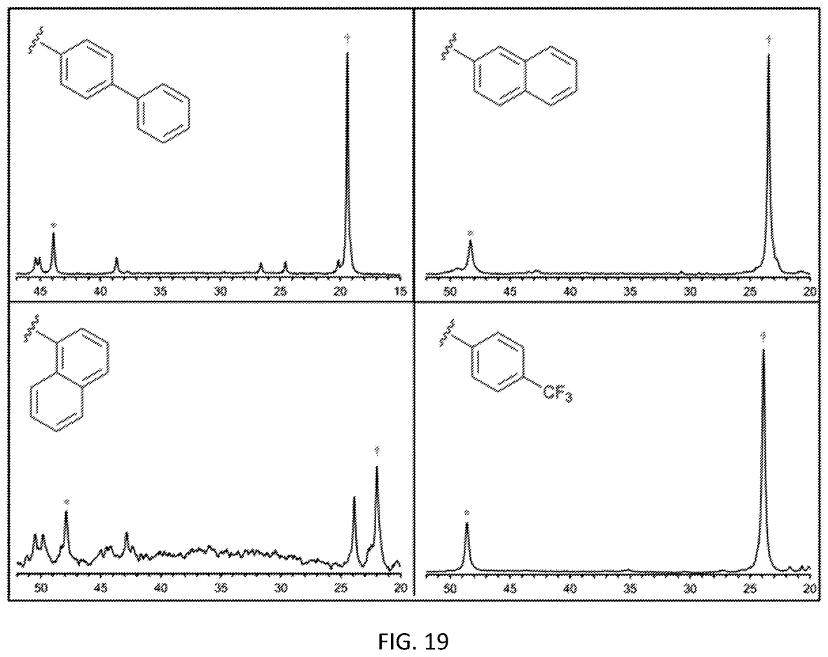

NMR spectra are shown in FIG. 19. The spectra are focused on the most shifted axial proton resonance. The resonances corresponding to the desired SAP (*) and TSAP (+) isomer of the RRRR-/SSSS-stereoisomers are labeled. The amount of other diastereomers differs based on the steric effects of the substituent. Variation in the extent of deracemization may arise from differences in both the sterics and electronics of the substituent.

In an alternative scheme, some compounds are prepared by a modified procedure using the chemical initiator AIBN in place of UV-irradiation. This alternative procedure may be useful when Ar is alkoxy-substituted phenyl, resulting in selective bromination primarily at the benzylic position as desired.

In another alternative scheme, when R¹ is an alkenyl group, the compound may be synthesized by functionalizing with a hydroxide instead of a halide. The use of MMPP introduces an α-hydroxyl, is then converted to a triflate, tosylate, or mesylate. In the exemplary scheme shown below, R¹ is —CH═CHC(O)OH and R is C₁-C₄ alkyl, as previously described; MMPP is magnesium monoperoxyphthalate.

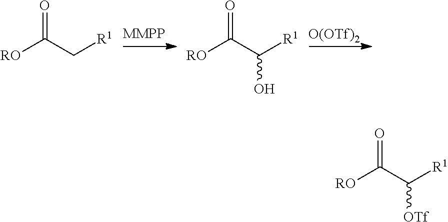

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A compound according to formula IB or IA, or a salt or stereoisomer thereof: 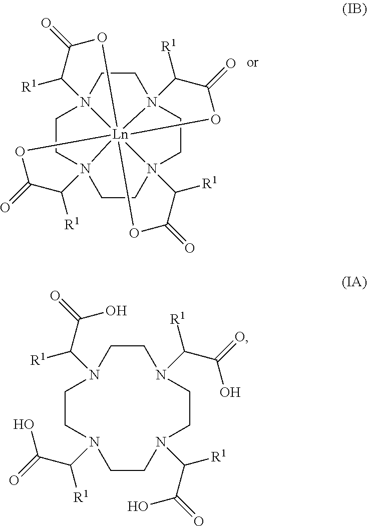
where
each R¹ independently is an aryl or aliphatic group having an sp²-hybridized or sp-hybridized carbon at its attachment point to the carbon atom alpha to nitrogen; and
Ln is a lanthanide ion.

2-3. (canceled)

4. The compound of claim 1, wherein the compound is optically active.

5. The compound of claim 1, wherein each R¹ independently is aryl, alkenyl, or alkynyl.

6. The compound of claim 1, wherein each R¹ independently is aryl substituted with one or more substituents R², where each R² independently is —(CH₂)ₙC(O)OR^{a}, —(CH₂)ₙOR^{a}, —(CH₂)ₙOC(O)R^{a}, —(CH₂)ₙC(O)R^{a}, —(CH₂)ₙC(S)R^{a}, —(CH₂)ₙCOR^{a}, —(CH₂)ₙCSR^{a}, —(CH₂)ₙSR^{a}, —(CH₂)ₙNO₂, —(CH₂)ₙN(R^{b})R^{c}, —(CH₂)ₙC(O)N(R^{b})R^{c}, —(CH₂)ₙC(S)N(R^{b})R^{c}, —(CH₂)ₙCN, alkyl, alkenyl, alkynyl, or halo, where each R^{a}, R^{b}, and R^{c} independently is H or alkyl, and n is an integer from 0 to 10.

7. The compound of claim 6, wherein n is 0.

8. (canceled)

9. The compound of claim 1, wherein each R¹ independently is 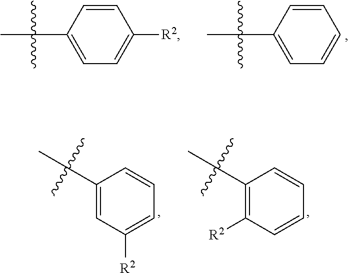
unsubstituted naphthalenyl, or alkenyl, wherein each R² independently is —C(O)OR^{a}, —OR^{a}, or alkyl.

10. (canceled)

11. The compound of claim 1, wherein each R¹ independently is 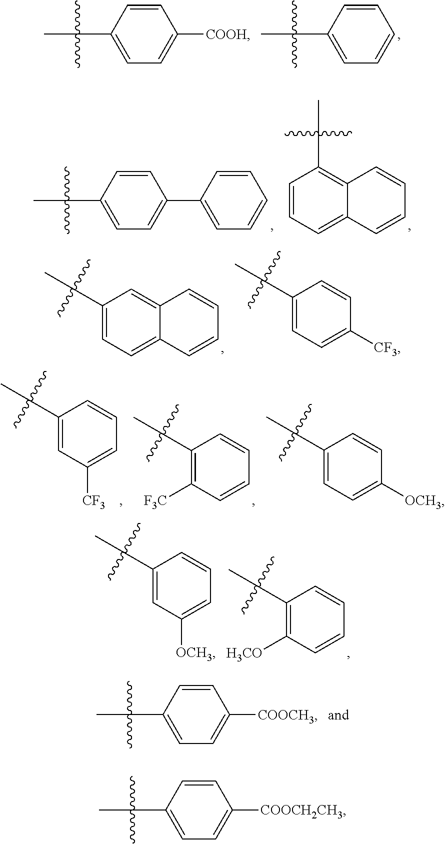
or —CH═CHCOOH.

12. The compound of claim 1, where each R¹ is the same.

13. (canceled)

14. The compound of claim 1, wherein the compound is:

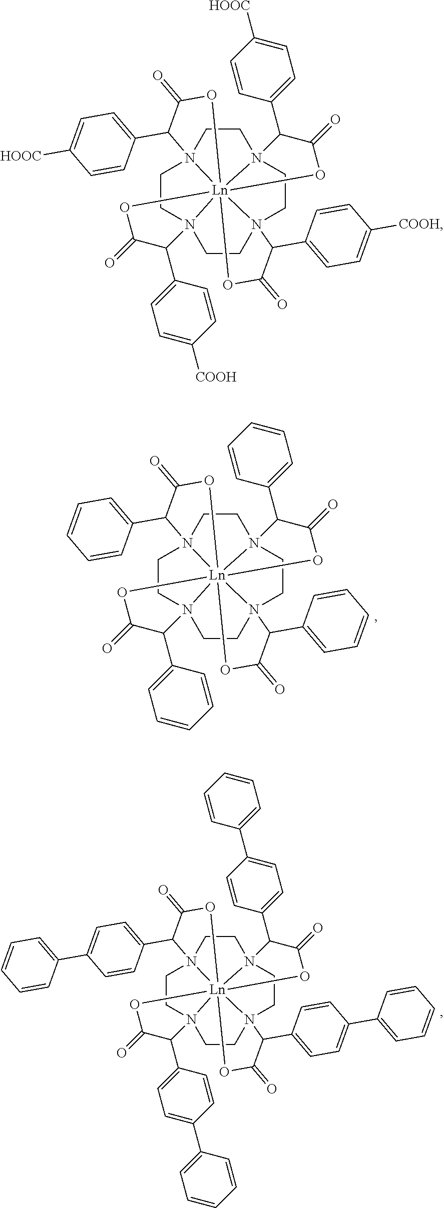

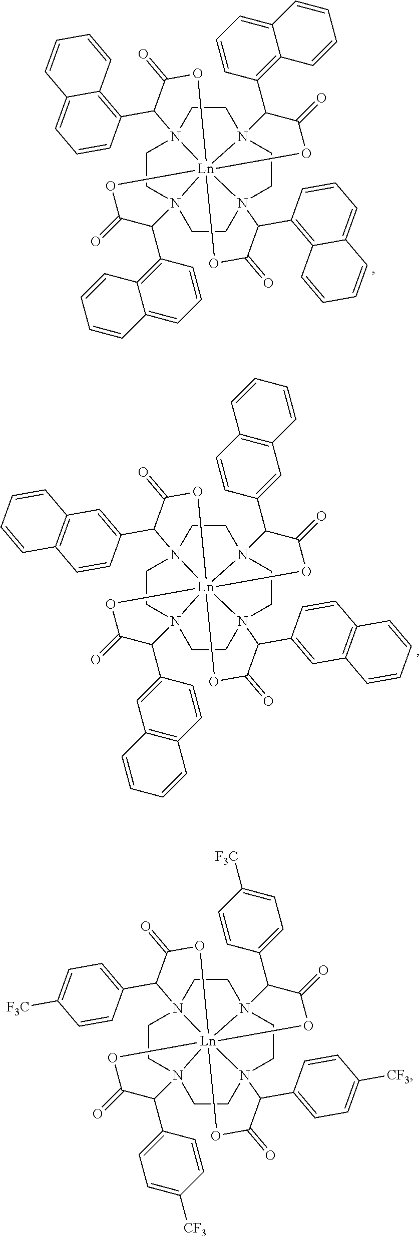

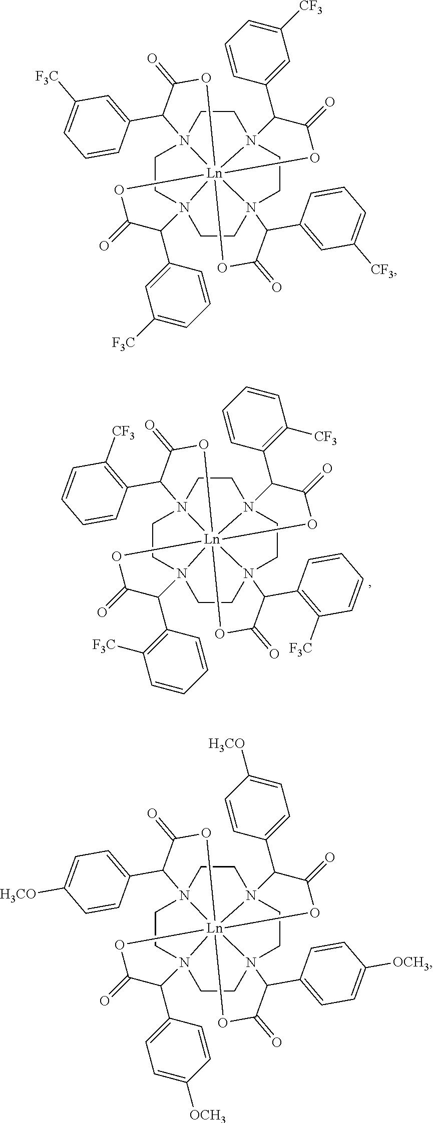

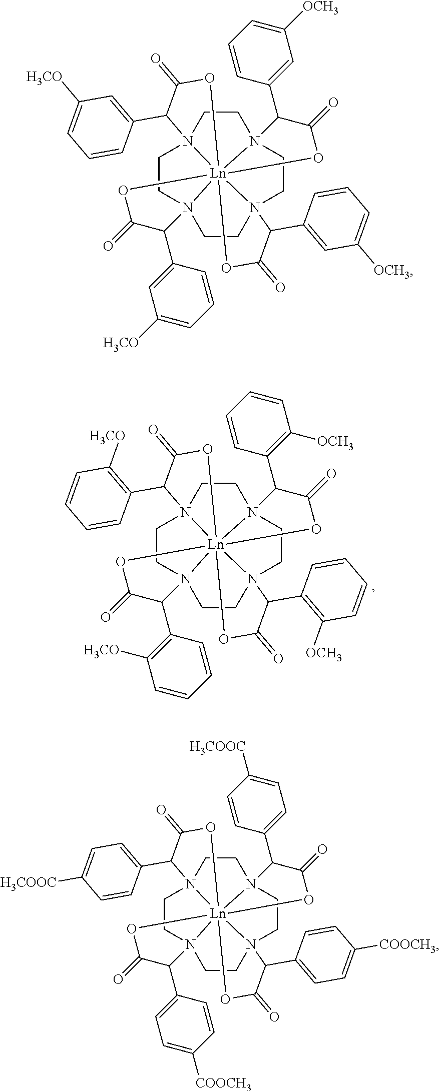

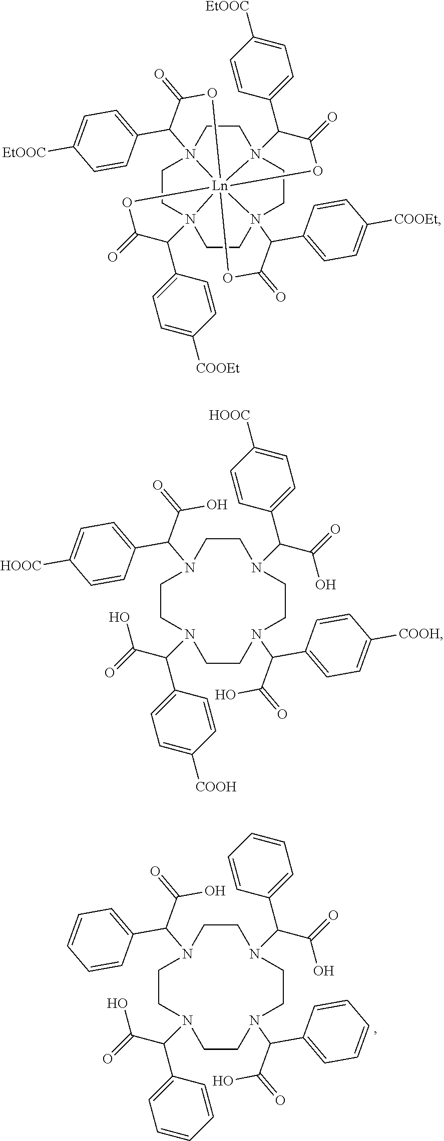

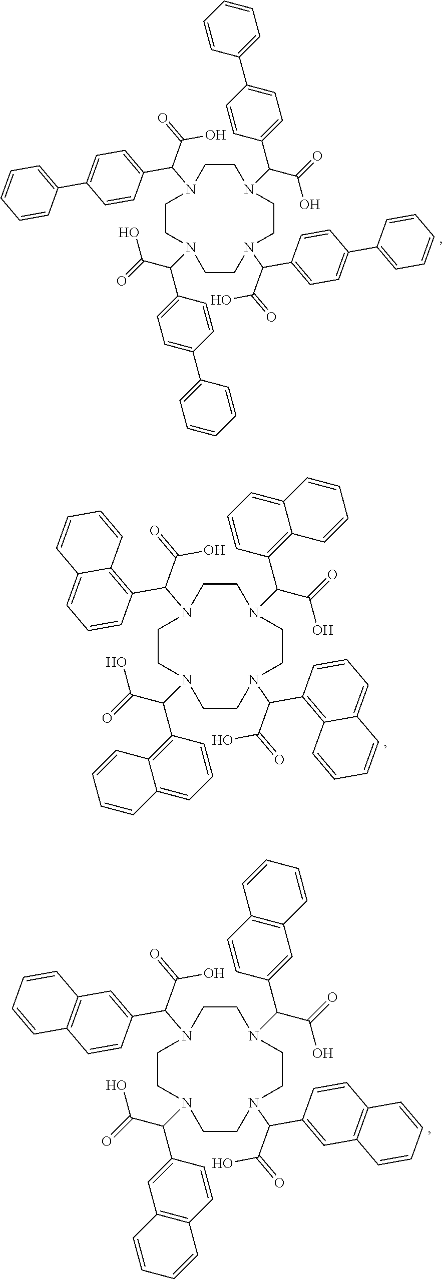 [SMILES: O=C(O)C(c1ccc(-c2ccccc2)cc1)N1CCN(C(C(=O)O)c2ccc(-c3ccccc3)cc2)CCN(C(C(=O)O)c2ccc(-c3ccccc3)cc2)CCN(C(C(=O)O)c2ccc(-c3ccccc3)cc2)CC1 ; O=C(O)C(c1cccc2ccccc12)N1CCN(C(C(=O)O)c2cccc3ccccc23)CCN(C(C(=O)O)c2cccc3ccccc23)CCN(C(C(=O)O)c2cccc3ccccc23)CC1 ; O=C(O)C(c1ccc2ccccc2c1)N1CCN(C(C(=O)O)c2ccc3ccccc3c2)CCN(C(C(=O)O)c2ccc3ccccc3c2)CCN(C(C(=O)O)c2ccc3ccccc3c2)CC1]

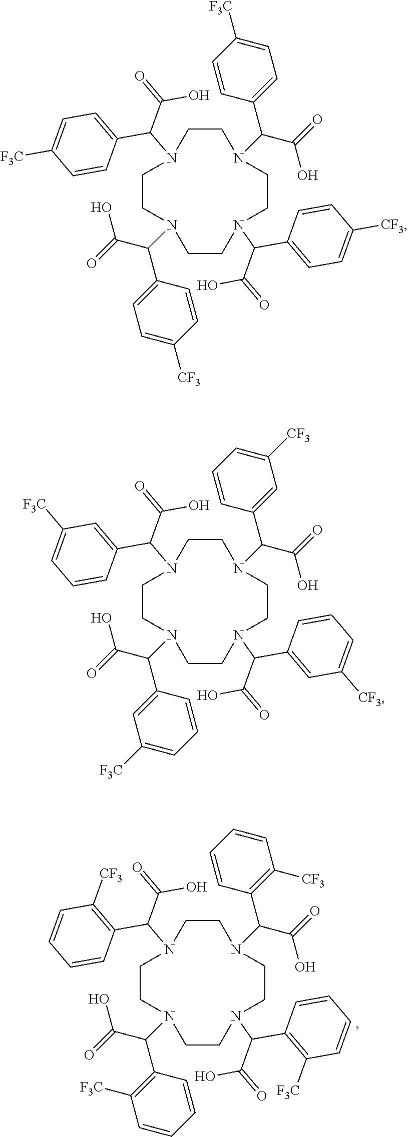 [SMILES: O=C(O)C(c1ccc(C(F)(F)F)cc1)N1CCN(C(C(=O)O)c2ccc(C(F)(F)F)cc2)CCN(C(C(=O)O)c2ccc(C(F)(F)F)cc2)CCN(C(C(=O)O)c2ccc(C(F)(F)F)cc2)CC1 ; O=C(O)C(c1cccc(C(F)(F)F)c1)N1CCN(C(C(=O)O)c2cccc(C(F)(F)F)c2)CCN(C(C(=O)O)c2cccc(C(F)(F)F)c2)CCN(C(C(=O)O)c2cccc(C(F)(F)F)c2)CC1 ; O=C(O)C(c1ccccc1C(F)(F)F)N1CCN(C(C(=O)O)c2ccccc2C(F)(F)F)CCN(C(C(=O)O)c2ccccc2C(F)(F)F)CCN(C(C(=O)O)c2ccccc2C(F)(F)F)CC1]

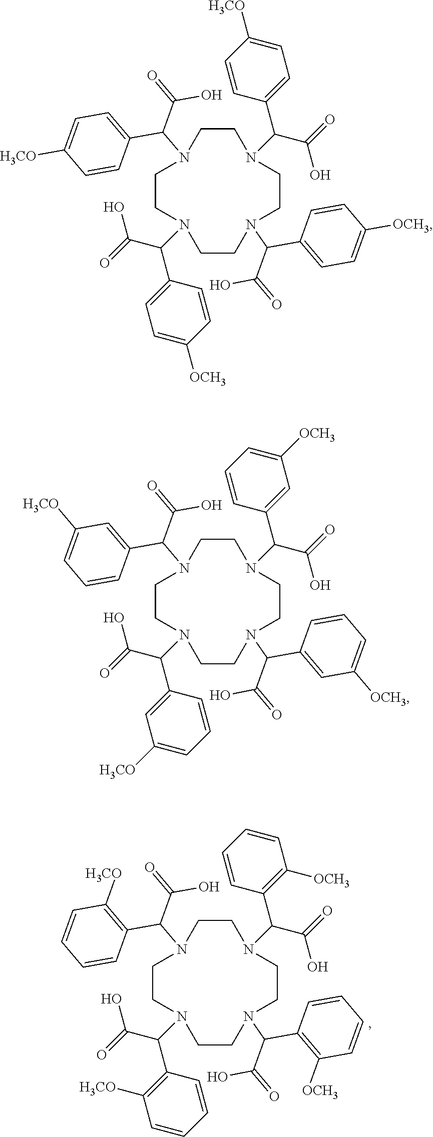 [SMILES: COc1ccc(C(C(=O)O)N2CCN(C(C(=O)O)c3ccc(OC)cc3)CCN(C(C(=O)O)c3ccc(OC)cc3)CCN(C(C(=O)O)c3ccc(OC)cc3)CC2)cc1 ; COc1cccc(C(C(=O)O)N2CCN(C(C(=O)O)c3cccc(OC)c3)CCN(C(C(=O)O)c3cccc(OC)c3)CCN(C(C(=O)O)c3cccc(OC)c3)CC2)c1 ; COc1ccccc1C(C(=O)O)N1CCN(C(C(=O)O)c2ccccc2OC)CCN(C(C(=O)O)c2ccccc2OC)CCN(C(C(=O)O)c2ccccc2OC)CC1]

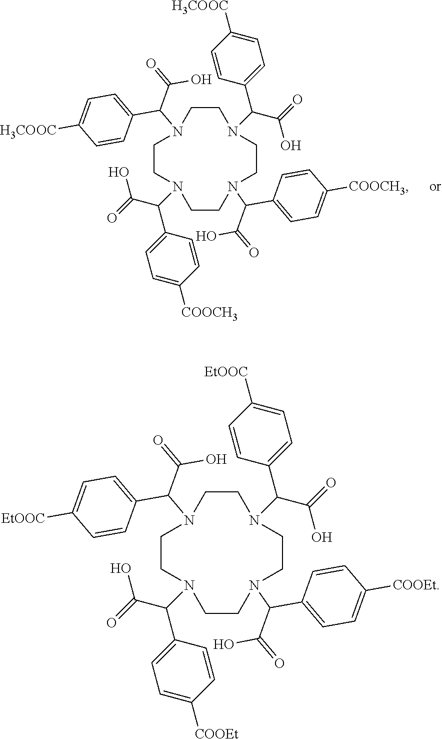

15. The compound according to claim 14, wherein the compound is

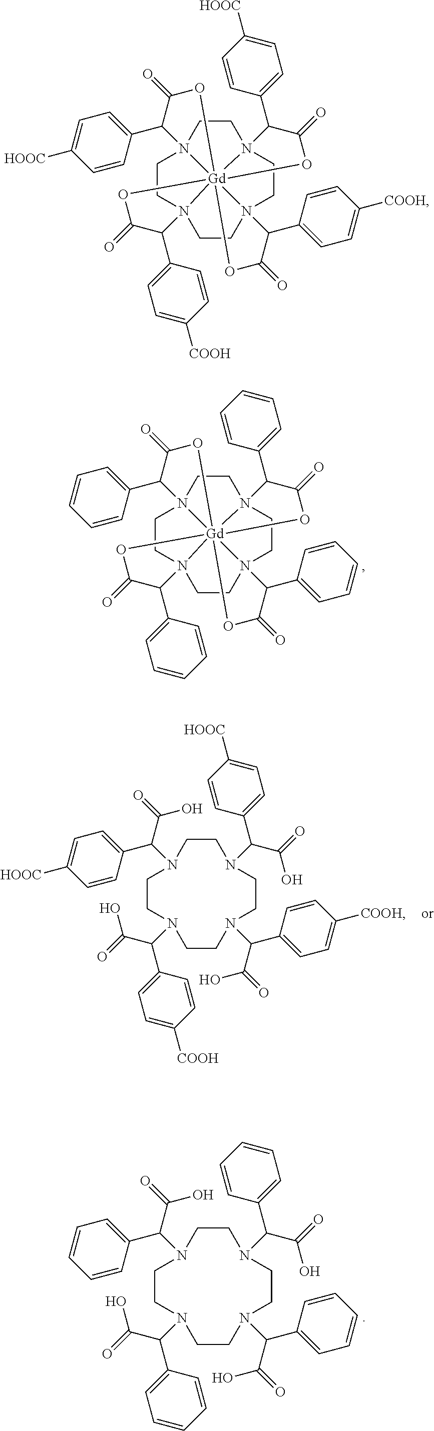

16. The compound according to claim 1, wherein the compound according to formula IB is diastereoisomer, and molecules of the diastereomer comprise a mixture of RRRR- and SSSS-enantiomers.

17. The compound of claim 16, wherein the compound is:

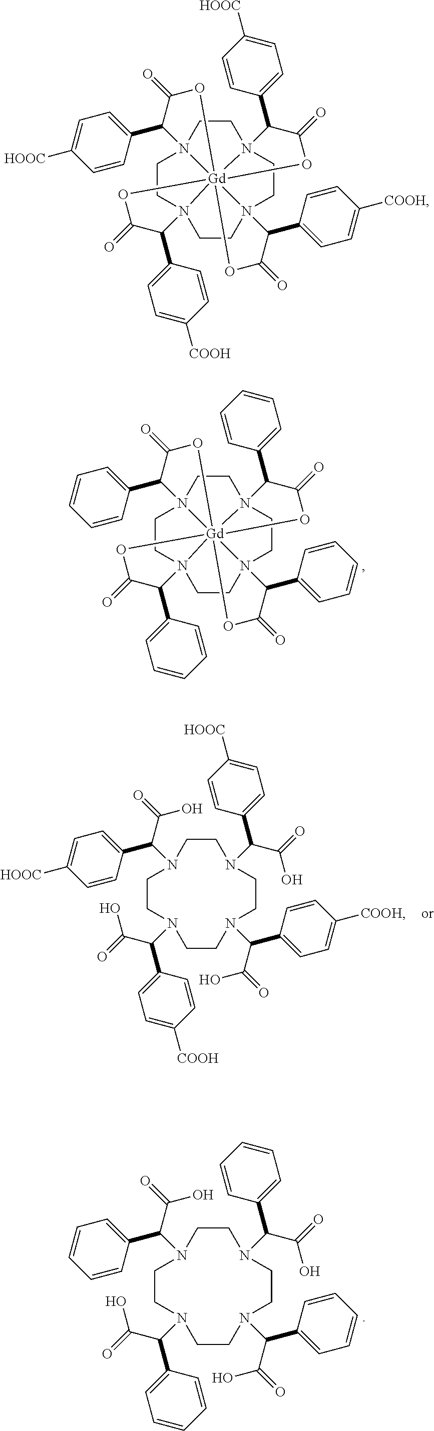

18. (canceled)

19. The compound according to claim 16, wherein:
at least 70% of molecules of the compound independently are RRRR- or SSSS-enantiomers; and
a solution of the compound in water has a relaxivity r₁≥6 mM⁻¹s⁻¹ at a nuclear magnetic resonance field strength of 0.5 T to 7 T and 310K.

20. (canceled)

21. A pharmaceutical composition comprising:
a compound according to claim 1 having a structure according to formula IB; and
a pharmaceutically acceptable carrier.

22. The pharmaceutical composition of claim 21, wherein the compound is a diastereomer and at least 70% of molecules of the compound independently are RRRR- or SSSS-enantiomers.

23. The pharmaceutical composition of claim 21, wherein the compound is 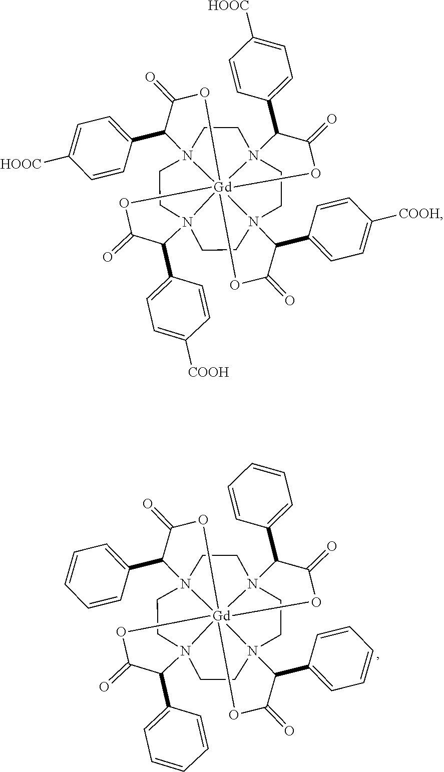
or a combination thereof.

24. (canceled)

25. A method for preparing a chelate according to formula IB, 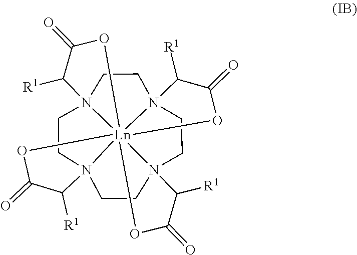
where R¹ is an aryl or aliphatic group having an sp²-hybridized or sp-hybridized carbon at its attachment point to the carbon atom alpha to nitrogen, comprising:
reacting an alkylating agent 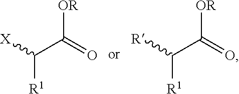
where R is C₁-C₄ alkyl, X is Br, Cl, or I, and R′ is triflate, tosylate, or mesylate, with cyclen, 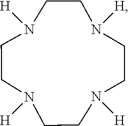
to provide an alkylated cyclen 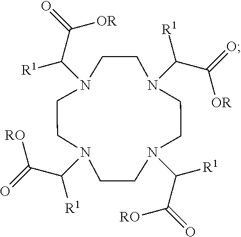
saponifying the alkylated cyclen to remove R and provide a compound according to Formula IA, 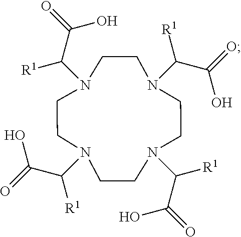
and
chelating a lanthanide (Ln) salt, oxide, or triflate with the compound according to Formula IA, 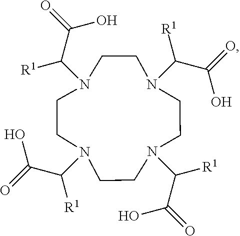
thereby providing a chelate according to Formula IB

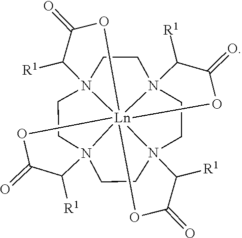

26-28. (canceled)

29. The method of claim 25, wherein:
(i) R¹ is 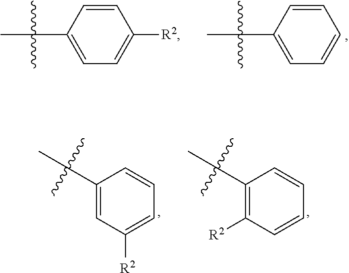
unsubstituted naphthalenyl, or
alkenyl, where R² is —COOR^{a} and R^{a} is C₁-C₃ alkyl or H; or
(ii) Ln is Gd, Eu, or a combination thereof; or
(iii) both (i) and (ii).

30-31. (canceled)

32. The method of claim 25, wherein:
(i) the chelate according to formula IB comprises a diastereomer, and molecules of the diastereomer comprise a mixture of RRRR- and SSSS-enantiomers; or
(ii) the chelate according to Formula IB is 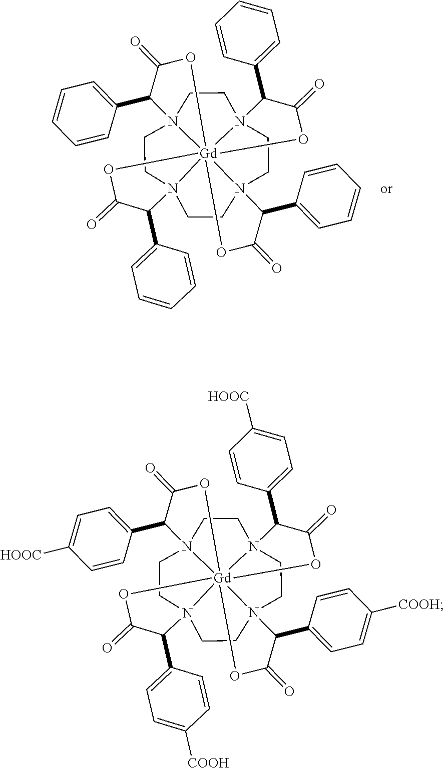
or
(iii) both (i) and (ii).

33. A method for magnetic resonance imaging, comprising:
administering to a subject an effective amount of a pharmaceutical composition according to claim 21; and
performing a magnetic resonance imaging procedure on the subject.

34-35. (canceled)
